(19) United States
(12) Patent Application Publication
(10) Pub. No.: US 2025/0109380 A1
(43) Pub. Date: Apr. 3, 2025

(54) METHOD FOR CULTURING NATURAL KILLER CELL, USING TRANSFORMED T CELL

(71) Applicant: GC Cell Corporation, Yongin-si Gyeonggi-do (KR)

(72) Inventors: Bokyung MIN, Yongin-si (KR); Gyeong Min PARK, Yongin-si (KR); Hyun Ah KIM, Yongin-si (KR); Bitna YANG, Yongin-si (KR); Yu Kyeong HWANG, Yongin-si (KR); Hyojin KIM, Yongin-si (KR)

(21) Appl. No.: 18/977,119

(22) Filed: Dec. 11, 2024

Related U.S. Application Data
(62) Division of application No. 16/613,601, filed on Nov. 14, 2019, now Pat. No. 12,203,065.

(30) Foreign Application Priority Data
May 26, 2017 (KR) 10-2017-0065180

(51) Int. Cl. C12N 5/0783; A61K 39/00

(52) U.S. Cl. CPC C12N 5/0638; A61K 40/15; A61K 40/42; C12N 5/0636; C12N 5/0646; C12N 2501/25; C12N 2502/1157; C12N 2510/00

(57) ABSTRACT

The present invention relates to a method for culturing natural killer cells, using genetically modified T cells. A method for culturing natural killer cells, using genetically modified T cells according to the present invention enables the effective proliferation and production of natural killer cells from a smaller amount of source cells. In addition, the method enhances the cytolytic activity of natural killer cells. Therefore, the method for culturing natural killer cells, using genetically modified T cells according to the present invention can find useful applications in commercializing cell therapy products. Further, the natural killer cells produced by the culturing method of the present invention can be useful as a cell therapy product.

### TECHNICAL FIELD

The present invention relates to a culture method for natural killer cells using transformed T cells.

### BACKGROUND ART

As a therapeutic method for the treatment of cancer patients and the prevention of recurrence of cancer, immunotherapy using the patient's immune function has been developed. In particular, immunotherapy using natural killer cells that can be mass-produced and frozen has been studied. Natural killer cells are lymphoid cells that account for about 15% of peripheral blood lymphocytes, and play an important role in the innate immune response.

Specifically, natural killer cells activate dendritic cells and induce cytotoxic T lymphocytes (CTLs) to specifically respond to tumors, thereby eliminating tumor cells. Natural killer cells directly kill malignant tumors such as sarcoma, myeloma, carcinoma, lymphoma, and leukemia. However, most natural killer cells present in the bodies of normal subjects exist in an inactive state, and activated natural killer cells are required to eliminate tumors. In addition, in a case of natural killer cells present in the bodies of cancer patients, the natural killer cells have functional defects due to the immune evasion mechanism of cancer cells.

Therefore, in order to use natural killer cells as a therapeutic agent, it is very important to activate the natural killer cells. In addition, since the number of natural killer cells present in the body is limited, it is essential to develop a technique for massively proliferating and freezing natural killer cells in the blood from normal subjects or patients.

As a method for massively proliferating natural killer cells, an ex vivo expansion culture method is used. For ex vivo expansion culture of natural killer cells, Peripheral blood mononuclear cells (PBMCs), CD3-cells, CD3-CD56+ cells, CD56+ cells, and the like are used as seed cells, and cytokines such as IL-2, IL-12, IL-15, and IL-21, LPS (Goodier et al., J. Immunol. 165(1):139-147, 2000), OKT-3 antibodies that stimulate CD3 (Condiotti et al., Experimental Hematol. 29(1):104-113, 2001) are used as proliferation factors for natural killer cells. Using the above-mentioned proliferation factors only, it is possible to proliferate natural killer cells about 3 to 10 times. However, with the above proliferation rate, it is difficult to commercialize natural killer cells into a therapeutic agent.

Recently, methods for proliferating natural killer cells using various types of feeder cells have been studied. Representative cell lines used as feeder cells include PBMC, EBV-LCL, and K562 cell line. K562 cell line is a blood cancer-derived cell line that lacks HLA and is a representative target cell line that natural killer cells can easily attack. The culture method using K562-based feeder cells for NK cells are as follows; a method in which cultures NK cells using 4-1BBL and membrane-bound IL-15 expressed K562 cell line, (Fujisaki et al., Cancer Res. 69(9):4010-4017, 2009), a method in which cultures NK cells using MICA, 4-1BBL, and IL-15 expressed K562 cell line (Gong et al., Tissue Antigens, 76(6)467-475, 2010), and a method in which cultures NK cells using 4-1BBL and membrane-bound IL-21 expressed K562 cell (Cecele JD et al, PloSONE, 7(1):e30264, 2012)

### DISCLOSURE OF INVENTION

### Technical Problem

Accordingly, in order to activate and proliferate natural killer cells, the present inventors have developed a method for effectively proliferating natural killer cells ex vivo, comprising expressing co-stimulatory molecules and growth factors, which can enhance proliferation of the natural killer cells, in CD4+ T cells.

Specifically, in order to increase efficiency of a culturing method for natural killer cells using the CD4(+) T cells as feeder cells, the present inventors have established transformed or genetically modified CD4(+) T cells. The present inventors have identified that in a case where the genetically modified CD4(+) T cells are co-cultured with peripheral blood mononuclear cells, such co-culture increases proliferation and abnormal cell-killing activity of natural killer cells, and thus have completed the present invention. Therefore, there is provided a culture method for proliferating natural killer cells in an efficient and stable manner using the genetically modified CD4(+) T cells.

### Solution to Problem

In an aspect of the present invention, there is provided a genetically modified or transformed CD4+ T cell that expresses at least one gene selected from the group consisting of 4-1BBL gene, mbIL-21 gene, OX40L gene, and mTNF-α gene.

In addition, in another aspect of the present invention, there is provided a culture method for natural killer cells, comprising a step of co-culturing the genetically modified CD4+ T cells and seed cells.

Furthermore, in yet another aspect of the present invention, there is provided a composition for culturing natural killer cells, comprising the genetically modified CD4+ T cell as an active ingredient.

In addition, in still yet another aspect of the present invention, there is provided a natural killer cell produced by the culture method.

### Advantageous Effects of Invention

The culture method for natural killer cells using the genetically modified T cell of the present invention allows natural killer cells to be effectively proliferated and produced from a small number of seed cells. In addition, the culture method also improves abnormal cell-killing activity of natural killer cells. Therefore, the culture method for natural killer cells using the genetically modified T cell of the present invention can be usefully used for commercialization into a cell therapeutic agent. Furthermore, a natural killer cell produced by the culture method of the present invention can be usefully used as a cell therapeutic agent.

### BRIEF DESCRIPTION OF DRAWINGS

FIG. 1a shows expression of single genes transduced into Hut78 cell line based on FACS.

FIG. 1b shows expression of double genes transduced into Hut78 cell line based on FACS.

FIG. 1c shows gene expression in Hut78 cell line based on FACS.

FIG. 1d shows expression of single genes transduced into Hut78 cell line based on FACS.

FIG. 1e shows expression of mTNF-α/OX40L and mTNF-α/4-1BBL double genes transduced into Hut78 cell line based on FACS.

FIG. 1f shows expression of mbIL-21/OX40L and mbIL-21/4-1BBL double genes transduced into Hut78 cell line based on FACS.

FIG. 1g shows expression of triple genes transduced into Hut78 cell line based on FACS.

FIG. 1h shows expression of quadruple genes transduced into Hut78 cell line based on FACS.

FIG. 2a shows expression of single genes transduced into H9 cell line based on FACS.

FIG. 2b shows expression of double genes transduced into H9 cell line based on FACS.

FIG. 3a shows expression of single genes transduced into Jurkat T cell line based on FACS.

FIG. 3b shows expression of double genes transduced into Jurkat T cell line based on FACS.

FIG. 4a shows fold increase, for each transduced gene, of natural killer cells produced by co-culturing Hut78 T cells having undergone single gene or double gene transduction, and peripheral blood mononuclear cells.

FIG. 4b shows fold increase, for each transduced gene, of natural killer cells produced by co-culturing H9 T cells having undergone single gene or double gene transduction, and peripheral blood mononuclear cells.

FIG. 4c shows fold increase, for each transduced gene, of natural killer cells produced by co-culturing Jurkat T cells having undergone single gene or double gene transduction, and peripheral blood mononuclear cells.

FIG. 4d shows fold increase, for each transduced gene, of natural killer cells produced by co-culturing Hut78 T cell line having undergone single gene to quadruple gene transduction, and peripheral blood mononuclear cells.

FIG. 5a shows fold increase of natural killer cells (D7R) produced by co-culturing Hut78 T cell line having undergone triple gene transduction, and peripheral blood mononuclear cells.

FIG. 5b shows fold increase, calculated as log values, of natural killer cells (D7R) produced by co-culturing Hut78 T cell line having undergone triple gene transduction, and peripheral blood mononuclear cells.

FIG. 5c shows fold increases of natural killer cells (D11R) produced by co-culturing Hut78 T cell line having undergone triple gene transduction, and peripheral blood mononuclear cells.

FIG. 5d shows fold increase, calculated as log values, of natural killer cells (D11R) produced by co-culturing Hut78 T cell line having undergone triple gene transduction, and peripheral blood mononuclear cells.

FIG. 6a shows viability, for each transduced gene, of natural killer cells produced by co-culturing Hut78 T cell line having undergone single gene or double gene transduction, and peripheral blood mononuclear cells.

FIG. 6b shows viability, for each transduced gene, of natural killer cells produced by co-culturing H9 T cell line having undergone single gene or double gene transduction, and peripheral blood mononuclear cells.

FIG. 6c shows viability, for each transduced gene, of natural killer cells produced by co-culturing Jurkat T cell line having undergone single gene or double gene transduction, and peripheral blood mononuclear cells.

FIG. 6d shows viability, for each transduced gene, of natural killer cells produced by co-culturing Hut78 T cell line having undergone single gene to quadruple gene transduction, and peripheral blood mononuclear cells.

FIG. 7a shows purity (CD3-CD56+), for each transduced gene, of natural killer cells produced by co-culturing Hut78 T cell line having undergone single gene or double gene transduction, and peripheral blood mononuclear cells.

FIG. 7b shows activation (CD16+CD56+), for each transduced gene, of natural killer cells produced by co-culturing Hut78 T cell line having undergone single gene or double gene transduction, and peripheral blood mononuclear cells.

FIG. 7c shows purity (CD3-CD56+), for each transduced gene, of natural killer cells produced by co-culturing H9 T cell line having undergone single gene or double gene transduction, and peripheral blood mononuclear cells.

FIG. 7d shows activation (CD16+CD56+), for each transduced gene, of natural killer cells produced by co-culturing H9 T cell line having undergone single gene or double gene transduction, and peripheral blood mononuclear cells.

FIG. 7e shows purity (CD3-CD56+), for each transduced gene, of natural killer cells produced by co-culturing Jurkat T cell line having undergone single gene or double gene transduction, and peripheral blood mononuclear cells.

FIG. 7f shows activation (CD16+CD56+), for each transduced gene, of natural killer cells produced by co-culturing Jurkat cell line having undergone single gene or double gene transduction, and peripheral blood mononuclear cells.

FIG. 7g shows purity (CD3-CD56+), for each transduced gene, of natural killer cells produced by co-culturing Hut78 T cell line having undergone single gene to quadruple gene transduction, and peripheral blood mononuclear cells.

FIG. 7h shows activation (CD16+CD56+), for each transduced gene, of natural killer cells produced by co-culturing Hut78 T cell line having undergone single gene to quadruple gene transduction, and peripheral blood mononuclear cells.

FIG. 8a shows expression levels of the phenotypic marker CD16, for each transduced gene, in natural killer cells produced by co-culturing Hut78 T cell line having undergone single gene or double gene transduction, and peripheral blood mononuclear cells.

FIG. 8b shows expression levels of the phenotypic marker NKG2D, for each transduced gene, in natural killer cells produced by co-culturing Hut78 T cell line having undergone single gene or double gene transduction, and peripheral blood mononuclear cells.

FIG. 8c shows expression levels of the phenotypic marker NKp30, for each transduced gene, in natural killer cells produced by co-culturing Hut78 T cell line having undergone single gene or double gene transduction, and peripheral blood mononuclear cells.

FIG. 8d shows expression levels of the phenotypic marker NKp44, for each transduced gene, in natural killer cells produced by co-culturing Hut78 T cell line having undergone single gene or double gene transduction, and peripheral blood mononuclear cells.

FIG. 8e shows expression levels of the phenotypic marker NKp46, for each transduced gene, in natural killer cells produced by co-culturing Hut78 T cell line having undergone single gene or double gene transduction, and peripheral blood mononuclear cells.

FIG. 8f shows expression levels of the phenotypic marker DNAM-1, for each transduced gene, in natural killer cells produced by co-culturing Hut78 T cell line having undergone single gene or double gene transduction, and peripheral blood mononuclear cells.

FIG. 8g shows expression levels of the phenotypic marker CXCR3, for each transduced gene, in natural killer cells produced by co-culturing Hut78 T cell line having undergone single gene or double gene transduction, and peripheral blood mononuclear cells.

FIG. 9a shows expression levels of the phenotypic marker CD16, for each transduced gene, in natural killer cells produced by co-culturing H9 T cell line having undergone single gene or double gene transduction, and peripheral blood mononuclear cells.

FIG. 9b shows expression levels of the phenotypic marker NKG2D, for each transduced gene, in natural killer cells produced by co-culturing H9 T cell line having undergone single gene or double gene transduction, and peripheral blood mononuclear cells.

FIG. 9c shows expression levels of the phenotypic marker NKp30, for each transduced gene, in natural killer cells produced by co-culturing H9 T cell line having undergone single gene or double gene transduction, and peripheral blood mononuclear cells.

FIG. 9d shows expression levels of the phenotypic marker NKp44, for each transduced gene, in natural killer cells produced by co-culturing H9 T cell line having undergone single gene or double gene transduction, and peripheral blood mononuclear cells.

FIG. 9e shows expression levels of the phenotypic marker NKp46, for each transduced gene, in natural killer cells produced by co-culturing H9 T cell line having undergone single gene or double gene transduction, and peripheral blood mononuclear cells.

FIG. 9f shows expression levels of the phenotypic marker DNAM-1, for each transduced gene, in natural killer cells produced by co-culturing H9 T cell line having undergone single gene or double gene transduction, and peripheral blood mononuclear cells.

FIG. 9g shows expression levels of the phenotypic marker CXCR3, for each transduced gene, in natural killer cells produced by co-culturing H9 T cell line having undergone single gene or double gene transduction, and peripheral blood mononuclear cells.

FIG. 10a shows expression levels of the phenotypic marker CD16, for each transduced gene, in natural killer cells produced by co-culturing Jurkat T cell line having undergone single gene or double gene transduction, and peripheral blood mononuclear cells.

FIG. 10b shows expression levels of the phenotypic marker NKG2D, for each transduced gene, in natural killer cells produced by co-culturing Jurkat T cell line having undergone single gene or double gene transduction, and peripheral blood mononuclear cells.

FIG. 10c shows expression levels of the phenotypic marker NKp30, for each transduced gene, in natural killer cells produced by co-culturing Jurkat T cell line having undergone single gene or double gene transduction, and peripheral blood mononuclear cells.

FIG. 10d shows expression levels of the phenotypic marker NKp44, for each transduced gene, in natural killer cells produced by co-culturing Jurkat T cell line having undergone single gene or double gene transduction, and peripheral blood mononuclear cells.

FIG. 10e shows expression levels of the phenotypic marker NKp46, for each transduced gene, in natural killer cells produced by co-culturing Jurkat T cell line having undergone single gene or double gene transduction, and peripheral blood mononuclear cells.

FIG. 10f shows expression levels of the phenotypic marker DNAM-1, for each transduced gene, in natural killer cells produced by co-culturing Jurkat T cell line having undergone single gene or double gene transduction, and peripheral blood mononuclear cells.

FIG. 10g shows expression levels of the phenotypic marker CXCR3, for each transduced gene, in natural killer cells produced by co-culturing Jurkat T cell line having undergone single gene or double gene transduction, and peripheral blood mononuclear cells.

FIG. 11a shows expression levels of the phenotypic marker CD16, for each transduced gene, in natural killer cells produced by co-culturing Hut78 T cell line having undergone single gene to quadruple gene transduction, and peripheral blood mononuclear cells.

FIG. 11b shows expression levels of the phenotypic marker NKG2A, for each transduced gene, in natural killer cells produced by co-culturing Hut78 T cell line having undergone single gene to quadruple gene transduction, and peripheral blood mononuclear cells.

FIG. 11c shows expression levels of the phenotypic marker NKG2C, for each transduced gene, in natural killer cells produced by co-culturing Hut78 T cell line having undergone single gene to quadruple gene transduction, and peripheral blood mononuclear cells.

FIG. 11d shows expression levels of the phenotypic marker NKG2D, for each transduced gene, in natural killer cells produced by co-culturing Hut78 T cell line having undergone single gene to quadruple gene transduction, and peripheral blood mononuclear cells.

FIG. 11e shows expression levels of the phenotypic marker NKp30, for each transduced gene, in natural killer cells produced by co-culturing Hut78 T cell line having undergone single gene to quadruple gene transduction, and peripheral blood mononuclear cells.

FIG. 11f shows expression levels of the phenotypic marker NKp44, for each transduced gene, in natural killer cells produced by co-culturing Hut78 T cell line having undergone single gene to quadruple gene transduction, and peripheral blood mononuclear cells.

FIG. 11g shows expression levels of the phenotypic marker NKp46, for each transduced gene, in natural killer cells produced by co-culturing Hut78 T cell line having undergone single gene to quadruple gene transduction, and peripheral blood mononuclear cells.

FIG. 11h shows expression levels of the phenotypic marker DNAM-1, for each transduced gene, in natural killer cells produced by co-culturing Hut78 T cell line having undergone single gene to quadruple gene transduction, and peripheral blood mononuclear cells.

FIG. 11i shows expression levels of the phenotypic marker CXCR3, for each transduced gene, in natural killer cells produced by co-culturing Hut78 T cell line having undergone single gene to quadruple gene transduction, and peripheral blood mononuclear cells.

FIG. 11j shows expression levels of the phenotypic marker CD62L, for each transduced gene, in natural killer cells produced by co-culturing Hut78 T cell line having undergone single gene to quadruple gene transduction, and peripheral blood mononuclear cells.

FIG. 11k shows expression levels of the phenotypic marker CD57, for each transduced gene, in natural killer cells produced by co-culturing Hut78 T cell line having undergone single gene to quadruple gene transduction, and peripheral blood mononuclear cells.

FIG. 11l shows expression levels of the phenotypic marker CD69, for each transduced gene, in natural killer cells produced by co-culturing Hut78 T cell line having undergone single gene to quadruple gene transduction, and peripheral blood mononuclear cells.

FIG. 12a shows expression levels of the phenotypic markers CD16, NKG2A, NKG2C, and NKG2D in natural killer cells produced by co-culturing Hut78 T cell line having undergone triple gene transduction, and peripheral blood mononuclear cells.

FIG. 12b shows expression levels of the phenotypic markers NKp30, NKp44, NKp46, and DNAM-1 in natural killer cells produced by co-culturing Hut78 T cell line having undergone triple gene transduction, and peripheral blood mononuclear cells.

FIG. 12c shows expression levels of the phenotypic markers CD62L, CD69, CXCR3, and CD57 in natural killer cells produced by co-culturing Hut78 T cell line having undergone triple gene transduction, and peripheral blood mononuclear cells.

FIG. 13a shows tumor cell-killing activity, for each transduced gene, of natural killer cells produced by co-culturing Hut78 T cell line having undergone single gene or double gene transduction, and peripheral blood mononuclear cells.

FIG. 13b shows tumor cell-killing activity, for each transduced gene, of natural killer cells produced by co-culturing H9 T cell line having undergone single gene or double gene transduction, and peripheral blood mononuclear cells.

FIG. 13c shows tumor cell-killing activity, for each transduced gene, of natural killer cells produced by co-culturing Jurkat T cell line having undergone single gene or double gene transduction, and peripheral blood mononuclear cells.

FIG. 14a shows tumor cell-killing activity (E:T=10:1), for each transduced gene, of natural killer cells produced by co-culturing Hut78 T cell line having undergone single gene to quadruple gene transduction, and peripheral blood mononuclear cells.

FIG. 14b shows tumor cell-killing activity (E:T=3:1), for each transduced gene, of natural killer cells produced by co-culturing Hut78 T cell line having undergone single gene to quadruple gene transduction, and peripheral blood mononuclear cells.

FIG. 14c shows tumor cell-killing activity (E:T=1:1), for each transduced gene, of natural killer cells produced by co-culturing Hut78 T cell line having undergone single gene to quadruple gene transduction, and peripheral blood mononuclear cells.

FIG. 14d shows tumor cell-killing activity (E:T=0.3:1), for each transduced gene, of natural killer cells produced by co-culturing Hut78 T cell line having undergone single gene to quadruple gene transduction, and peripheral blood mononuclear cells.

FIG. 15a shows tumor cell-killing activity, for respective cell ratios, of natural killer cells (NoS1) produced by co-culturing Hut78 T cell line having undergone triple gene transduction, and peripheral blood mononuclear cells.

FIG. 15b shows tumor cell-killing activity, for respective cell ratios, of natural killer cells (D7RS5) produced by co-culturing Hut78 T cell line having undergone triple gene transduction, and peripheral blood mononuclear cells.

FIG. 15c shows tumor cell-killing activity, for respective cell ratios, of natural killer cells (D11RS4) produced by co-culturing Hut78 T cell line having undergone triple gene transduction, and peripheral blood mononuclear cells.

FIG. 16a shows, expression levels of the proliferation marker Ki-67, for each transduced gene, in natural killer cells produced by co-culturing Hut78 T cell line having undergone single gene to quadruple gene transduction, and peripheral blood mononuclear cells.

FIG. 16b shows expression levels of the proliferation marker Ki-67 on day 16, for each transduced gene, in natural killer cells produced by co-culturing Hut78 T cell line having undergone single gene to quadruple gene transduction, and peripheral blood mononuclear cells.

FIG. 16c shows expression levels of the proliferation marker Ki-67 for each transduced gene, in terms of mean fluorescence index (MFI) ratio values, in natural killer cells produced by co-culturing Hut78 T cell line having undergone single gene to quadruple gene transduction, and peripheral blood mononuclear cells.

FIG. 16d shows expression levels of the proliferation marker Ki-67 for each transduced gene, in terms of mean fluorescence index ratio values, in natural killer cells produced by co-culturing Hut78 T cell line having undergone single gene to quadruple gene transduction, and peripheral blood mononuclear cells.

FIG. 17a shows expression levels of the proliferation marker Ki-67 in natural killer cells (D7R) produced by performing re-stimulation at 7-day intervals when co-culturing Hut78 T cell line having undergone triple gene transduction, and peripheral blood mononuclear cells.

FIG. 17b shows expression levels of the proliferation marker Ki-67 in natural killer cells (D11R) produced by performing re-stimulation at 11-day intervals when co-culturing Hut78 T cell line having undergone triple gene transduction, and peripheral blood mononuclear cells.

FIG. 17c shows expression levels of the proliferation marker Ki-67, in terms of mean fluorescence index ratio values, in natural killer cells (D7R) produced by performing re-stimulation at 7-day intervals when co-culturing Hut78 T cell line having undergone triple gene transduction, and peripheral blood mononuclear cells.

FIG. 17d shows expression levels of the proliferation marker Ki-67, in terms of mean fluorescence index ratio values, in natural killer cells (D11R) produced by performing re-stimulation at 11-day intervals when co-culturing Hut78 T cell line having undergone triple gene transduction, and peripheral blood mononuclear cells.

FIG. 18a shows expression levels of the co-stimulatory molecule CD25, for each transduced gene, in natural killer cells produced by co-culturing Hut78 T cell line having undergone single gene to quadruple gene transduction, and peripheral blood mononuclear cells.

FIG. 18b shows expression levels of the co-stimulatory molecule CD25 on day 11, for each transduced gene, in natural killer cells produced by co-culturing Hut78 T cell line having undergone single gene to quadruple gene transduction, and peripheral blood mononuclear cells.

FIG. 18c shows expression levels of the co-stimulatory molecule TNFRII, for each transduced gene, in natural killer cells produced by co-culturing Hut78 T cell line having undergone single gene to quadruple gene transduction, and peripheral blood mononuclear cells.

FIG. 18d shows expression levels of the co-stimulatory molecule TNFRII on day 16, for each transduced gene, in natural killer cells produced by co-culturing Hut78 T cell line having undergone single gene to quadruple gene transduction, and peripheral blood mononuclear cells.

FIG. 18e shows expression levels of the co-stimulatory molecule 41BB, for each transduced gene, in natural killer cells produced by co-culturing Hut78 T cell line having undergone single gene to quadruple gene transduction, and peripheral blood mononuclear cells.

FIG. 18f shows expression levels of the co-stimulatory molecule 41BB on day 7, for each transduced gene, in natural killer cells produced by co-culturing Hut78 T cell line having undergone single gene to quadruple gene transduction, and peripheral blood mononuclear cells.

FIG. 19a shows expression levels of the co-stimulatory molecule CD25 in natural killer cells (D7R) produced by performing re-stimulation at 7-day intervals when co-culturing Hut78 T cell line having undergone triple gene transduction, and peripheral blood mononuclear cells.

FIG. 19b shows expression levels of the co-stimulatory molecule TNFRII in natural killer cells (D7R) produced by performing re-stimulation at 7-day intervals when co-culturing Hut78 T cell line having undergone triple gene transduction, and peripheral blood mononuclear cells.

FIG. 19c shows expression levels of the co-stimulatory molecule 41BB in natural killer cells (D7R) produced by performing re-stimulation at 7-day intervals when co-culturing Hut78 T cell line having undergone triple gene transduction, and peripheral blood mononuclear cells.

FIG. 19d shows expression levels of the co-stimulatory molecule CD25 in natural killer cells (D11R) produced by performing re-stimulation at 11-day intervals when co-culturing Hut78 T cell line having undergone triple gene transduction, and peripheral blood mononuclear cells.

FIG. 19e shows expression levels of the co-stimulatory molecule TNFRII in natural killer cells (D11R) produced by performing re-stimulation at 11-day intervals when co-culturing Hut78 T cell line having undergone triple gene transduction, and peripheral blood mononuclear cells.

FIG. 19f shows expression levels of the co-stimulatory molecule 41BB in natural killer cells (D11R) produced by performing re-stimulation at 11-day intervals when co-culturing Hut78 T cell line having undergone triple gene transduction, and peripheral blood mononuclear cells.

FIG. 20a shows expression levels of CD107a, for each transduced gene, in natural killer cells produced by co-culturing Hut78 T cell line having undergone single gene to quadruple gene transduction, and peripheral blood mononuclear cells.

FIG. 20b shows expression levels of IFN-γ, for each transduced gene, in natural killer cells produced by co-culturing Hut78 T cell line having undergone single gene to quadruple gene transduction, and peripheral blood mononuclear cells.

FIG. 20c shows expression levels of TNF-α, for each transduced gene, in natural killer cells produced by co-culturing Hut78 T cell line having undergone single gene to quadruple gene transduction, and peripheral blood mononuclear cells.

FIG. 21a shows expression levels of CD107a, IFN-γ, and TNF-α in natural killer cells (NoS1) produced by co-culturing Hut78 T cell line having undergone triple gene transduction, and peripheral blood mononuclear cells.

FIG. 21b shows expression levels of CD107a, IFN-γ, and TNF-α in natural killer cells (D7RS5) produced by co-culturing Hut78 T cell line having undergone triple gene transduction, and peripheral blood mononuclear cells.

FIG. 21c shows expression levels of CD107a, IFN-γ, and TNF-α in natural killer cells (D11RS4) produced by co-culturing Hut78 T cell line having undergone triple gene transduction, and peripheral blood mononuclear cells.

### Best Mode for Carrying out the Invention

Hereinafter, the present invention will be described in detail.

In an aspect of the present invention, there is provided a transformed CD4+ T cell that expresses at least one gene selected from the group consisting of 4-1BBL gene, mbIL-21 gene, OX40L gene, and mTNF-α gene.

Specifically, when one gene is transduced, the gene may be 4-1BBL, mbIL-21, OX40L, or mTNF-α. In addition, when two genes are transduced, a combination of the genes may be mbIL-21/4-1BBL, 4-1BBL/OX40L, mTNF-α/4-1BBL, mbIL-21/OX40L, mbIL-21/mTNF-α, or mTNF-α/OX40L. In an embodiment of the present invention, combinations of genes, mbIL-21/4-1BBL, mTNF-α/OX40L, mTNF-α/4-1BBL, and mbIL-21/OX40L were transduced into T cells.

In addition, when three genes are transduced, a combination of the genes may be 4-1BBL/mbIL-21/OX40L, mbIL-21/OX40L/mTNF-α, mTNF-α/mbIL-21/4-1BBL, or 4-1BBL/OX40L/mTNF-α. In an embodiment of the present invention, a combination of genes, mTNF-α/mbIL-21/4-1BBL was transduced into T cells.

In addition, when four genes are transduced, a combination of the genes may be mTNF-α/mbIL-21/OX40L/4-1BBL. In an embodiment of the present invention, a combination of genes, mTNF-α/mbIL-21/OX40L/4-1BBL was transduced into T cells.

The term “4-1BBL” as used herein refers to one of TNF superfamily (TNFSF) called CD137L, and means a ligand that forms a trimer and binds to 4-1BB as a receptor. The 4-1BBL, gene may be derived from human.

Specifically, the 4-1BBL gene may be NCBI Reference Sequence: NM_003811, but is not limited thereto. The 4-1BBL gene may have a nucleotide sequence encoding the amino acid sequence represented by SEQ ID NO: 1. The nucleotide sequence encoding the amino acid sequence represented by SEQ ID NO: 1 may be the nucleotide sequence represented by SEQ ID NO: 2.

The term “mbIL-21” as used herein may be IL-21 designed to be bound to a cell membrane. Here, mbIL-21 may be a fusion protein formed by binding between IL-21 and a transmembrane protein. The transmembrane protein may be CD8α. Specifically, the transmembrane protein may be a transmembrane domain of CD8α.

Specifically, the IL-21 gene may be NCBI Reference Sequence: NM_021803.3, but is not limited thereto. In addition, the CD8α gene may be NCBI Reference Sequence: NM_001768, but is not limited thereto. The mbIL-21 is expressed in the form of IL-21 bound to a cell membrane. In addition, the mbIL-21 gene may have a nucleotide sequence encoding the amino acid sequence represented by SEQ ID NO: 3. The nucleotide sequence encoding the amino acid sequence represented by SEQ ID NO: 3 may be the nucleotide sequence represented by SEQ ID NO: 4.

The term “OX40L” as used herein is also referred to as ACT-4 receptor, TNF4-human, GP34, or CD134L, and means a ligand that binds to OX40. Specifically, the OX40L gene may be NCBI Reference Sequence: NM_003326, but is not limited thereto. The OX40L gene may have a nucleotide sequence encoding the amino acid sequence represented by SEQ ID NO: 5. The nucleotide sequence encoding the amino acid sequence represented by SEQ ID NO: 5 may be the nucleotide sequence represented by SEQ ID NO: 6.

The term “mTNF-α” as used herein means a gene obtained by causing a point mutation to occur on DNA of tumor necrosis factor-alpha so that alanine-valine, a site recognized by tumor necrosis factor-alpha-converting enzyme (TACE) in the amino acid sequence of tumor necrosis factor-alpha, is changed to proline-valine. Mutation of alanine to proline is randomly selected.

Specifically, the mTNF-α gene may have a nucleotide sequence encoding the amino acid sequence represented by SEQ ID NO: 8. The nucleotide sequence encoding the amino acid sequence represented by SEQ ID NO: 8 may be the nucleotide sequence represented by SEQ ID NO: 9.

The 4-1BBL gene, mbIL-21 gene, OX40L gene, or mTNF-α gene may be transduced through a recombinant lentivirus. However, transduction is not limited thereto.

As a method of transfecting the gene into cells, a biochemical method, a physical method, or a virus-mediated transfection method may be used. In addition, as the biochemical method, FuGene6 (Roche, USA), lipofectamine (Lipofectamine™ 2000, Invitrogen, USA), or ExGen 500 (MBI Fermentas International Inc., CANADA) may be used. In addition, a lipid-mediated method using lipofectamine may be used.

The term “vector” as used herein is an expression vector capable of expressing a gene of interest in a cell into which the vector is transduced, and refers to a gene construct containing an essential regulatory element operably linked so that a gene insert transduced into the vector is expressed.

In addition, as the expression vector containing the gene, any expression vector capable of expressing the gene in CD4+ T cell line may be used, and pCDH-CMV-MCS-EF1-Puro (SBI, CD510B-1) or pCDH-CMV-MCS-EF1-Neo (SBI, CD514B-1) lentiviral vector was used in a specific embodiment of the present invention.

The lentivirus means a virus which belongs to a retrovirus family and is characterized by a long latency period. The lentivirus can deliver genetic information into DNA of a host cell. Use of the lentivirus is one of the most effective methods of using gene delivery vectors that can replicate in non-dividing cells.

The CD4+ T cells may be ex vivo isolated CD4+ T cells, ex vivo expansion-cultured CD4+ T cells, or CD4+ T cell line (T lymphoma cell line). In addition, the CD4+ T cells may be auxiliary T cells, and may be a hybridoma obtained by fusing CD4+ T cells with cancer cells. Specifically, the CD4+ T cells may be any one selected from the group consisting of Hut78, H9, Jurkat, Loucy, Molt-3, Molt-13, PEER, RPMI8402, and TALL-01 cells. Hut78, H9, or Jurkat T cells may be preferred.

In another aspect of the present invention, there is provided a culture method for natural killer cells, comprising a step of co-culturing the transformed CD4+ T cells and seed cells.

The term “feeder cell” as used herein is also referred to as an auxiliary cell for culture, and means a cell which does not proliferate but has metabolic activity so that the cell produces various metabolites and thus helps proliferation of a target cell. The feeder cell may be a transformed CD4+ T cell that expresses at least one gene selected from the group consisting of 4-1BBL gene, mbIL-21 gene, OX40L gene, and mTNF-α gene.

T cells used as the feeder cells may be inactivated cells whose division/proliferation is inhibited or cells which have not been inactivated. Preferably, the T cells may be inactivated to ensure safety. As an inactivation method, a conventional method known in the art may be used. For example, a gamma ray-irradiation method may be used. In a case where T cells which have not been inactivated are used, most of them are tumor cells, and thus may be killed by activated natural killer cells during culture.

The term “seed cell” as used herein means a cell capable of proliferating to natural killer cells through appropriate culture. Specifically, the seed cells may be any one selected from the group consisting of peripheral blood, peripheral blood leukocytes, peripheral blood mononuclear cells (PBMCs), enriched natural killer cells, and isolated natural killer cells. The seed cells may preferably be, but are not limited to, CD3(−) cells from which CD3(+) cells have been eliminated.

In a culture method for natural killer cells, culture may be performed by mixing the feeder cells and the seed cells at a ratio of 0.1 or higher. Specifically, the feeder cells and the seed cells may be at a ratio of 0.1:1 to 50:1. More specifically, the ratio may be 0.5:1 to 40:1.

Even more specifically, the ratio may be 1:1 to 30:1. Most specifically, the ratio may be 2:1 to 20:1. In an embodiment, the feeder cells and the seed cells may be at a ratio of 5:1, but is not particularly limited thereto. The “ratio” means a ratio based on the number of cells.

In the culture method for natural killer cells, the seed cells may be mixed once with the feeder cells and culture may be performed for 5 days to 60 days, or may be mixed two or more times with the feeder cells and culture may be performed for 60 days or longer. Preferably, the seed cells may be mixed once with the feeder cells and culture was performed for 14 days to 21 days. However, the culture method is not limited thereto.

In the culture method for natural killer cells, natural killer cells and T lymphoma cell line are co-cultured in conventional animal cell culture medium such as AIM-V medium, RPMI1640, CellGro SCGM, X-VIVO20, IMDM, and DMEM. In co-culture, culture may be performed with addition of an antibody that has low affinity for T cells and stimulates T cells, and an interleukin. However, the culture method is not limited thereto.

The term “antibody which has low affinity for T cells and stimulates T cells” as used herein means a protein that specifically responds to CD3 antigens, a group of molecules each of which associates with T cell receptor (TCR) to form an antigen recognition complex. As compared with TCR, the CD3 molecule has a longer intracellular region and plays a role in delivering an antigen recognition signal into a cell.

The antibody that has low affinity for T cells and stimulates T cells, which can be used in the present invention, may preferably be an anti-CD3 antibody. Specifically, the anti-CD3 antibody may be OKT-3, UCHT1, or HIT3a.

The term “interleukin (IL)” as used herein refers to a group belonging to cytokines, and means a proteinaceous biologically-active substance produced by immunocompetent cells such as lymphocytes, monocytes, and macrophages. The interleukin may be IL-2, IL-15, IL-12, IL-18, or IL-21.

In an embodiment of the present invention, culture was performed with addition of OKT-3 antibody and IL-2. A concentration of the OKT-3 antibody to be added may be 0.1 ng/ml to 100 ng/ml. Preferably, the concentration of the OKT-3 antibody may be 10 ng/μl. A concentration of IL-2 may be 10 U/ml to 2,000 U/ml. Preferably, the concentration of IL-2 may be 500 U/ml. In addition, serum or plasma and an additional growth factor that supports growth of lymphocytes may be added and culture may be performed. The type of serum or plasma to be added to the medium is not particularly limited, and a variety of commercially available animal-derived scrum or plasma may be used. Preferably, human-derived serum or plasma, in particular, self-derived serum or plasma may be used.

The term “culture” as used herein means a method of growing cells under an environmental condition that is appropriately artificially regulated. The method of culturing the genetically modified CD4+ T cells may be carried out using methods well known in the art. Specifically, the culture may be performed in a batch or fed-batch process, or may be continuously performed in a repeated fed-batch process.

In addition, precursors suitable for the culture medium may be used. The above-mentioned raw materials may be added to a culture, during a culture process, in a batch, fed-batch, or continuous manner by an appropriate method. However, the present invention is not particularly limited thereto. A PH of the culture can be regulated by using, in an appropriate manner, basic compounds such as sodium hydroxide, potassium hydroxide, and ammonia, or acidic compounds such as phosphoric acid or sulfuric acid.

The culture method using the T cells as feeder cells allows natural killer cells to be selectively cultured from seed cells such as PBMCs, and enables a stable culture due to the fact that there is no difference in proliferation of natural killer cells as compared with a case where PBMC feeder cells are used depending on donors. Therefore, a large amount of natural killer cell therapeutic agents can be efficiently and stably obtained.

In addition, in yet another aspect of the present invention, there is provided a composition for culturing natural killer cells, comprising the genetically modified CD4+ T cell as an active ingredient.

Furthermore, in still yet another aspect of the present invention, there is provided a natural killer cell produced by the culture method for natural killer cells.

The natural killer cells cultured according to the culture method for natural killer cells can be frozen and do not exhibit impaired cell function even in a case of being thawed again. In addition, due to high expression of an activating receptor such as NKp46, the natural killer cells exhibit increased killing activity against a tumor cell line and increased cytokine secretion, and thus an excellent anticancer effect can be expected. Therefore, it is possible to prepare a cell therapeutic agent effective for tumor treatment, using a large number of activated natural killer cells which can be clinically applied.

In addition, in a composition for the prevention or treatment of an infectious disease, comprising, as an active ingredient, the natural killer cell produced by the culture method for natural killer cells, the natural killer cell may be contained in an amount of 10% to 95% by weight with respect to the total weight of the composition. In addition, the composition for the prevention or treatment of an infectious disease of the present invention may further comprise, in addition to the active ingredient, one or more active ingredients that exhibit the same or similar functions.

The composition for the prevention or treatment of an infectious disease may be formulated into a pharmaceutical composition for administration by further including one or more pharmaceutically acceptable carriers in addition to the above-mentioned active ingredients.

A dose of the pharmaceutical composition for the prevention or treatment of an infectious disease may be regulated depending on various factors including type of disease, severity of disease, types and amounts of active ingredients and other ingredients included in the composition, type of formulation and the patient's age, weight, general health condition, sex, and diet, time of administration, route of administration and secretion rate of composition, duration of treatment, and simultaneously used drugs. However, for desired effects, a dose of the natural killer cells according to the present invention may be 0.01×10⁷ cells/kg to 1.0×10⁹ cells/kg, and may be 0.5×10⁷ cells/kg to 1.0×10⁸ cells/kg. Here, the dose may be administered once a day or may be divided into several times a day.

In addition, the pharmaceutical composition for the prevention or treatment of an infectious disease may be administered to an individual by various methods known in the art. The route of administration may be appropriately selected by those skilled in the art in consideration of administration method, body fluid volume, viscosity, and the like.

### Mode for the Invention

Hereinafter, the present invention will be described in detail by way of examples. However, the following examples are only for illustrating the present invention, and the present invention is not limited to the following examples.

### Example 1. Construction of Recombinant Lentivirus

### Example 1.1. Construction of Recombinant Lentiviral Vector

As a lentiviral vector, pCDH-CMV-MCS-EF1-Puro (SBI, CD510B-1) or pCDH-CMV-MCS-EF1-Neo (SBI, CD514B-1) was used. As genes for transduction, 4-1BBL (TNF superfamily member 9, TNFSF9), mbIL-21 (membrane-bound IL-21), OX40L (TNF superfamily member 4 (TNFSF4) transcript variant 1) and mTNF-α (membrane-bound TNF alpha) genes were used.

Specifically, for the 4-1BBL gene (SEQ ID NO: 2), 4-1BBL gene expression vector (OriGene, RC211160) was used. For the mbIL-21 gene (SEQ ID NO: 4), pcDNA3.1 vector (GenScript, US) into which a codon-optimized mbIL-21 gene sequence is inserted was used. For the OX40L gene (SEQ ID NO: 6), a request for synthesis thereof was made to BIONEER CORPORATION.

For the mTNF-α gene (SEQ ID NO: 9), RNA was extracted from peripheral blood mononuclear cells (PBMCs), and then CDS was obtained therefrom by reverse transcriptase (RT)-PCR. In order for TNF-α to be secreted, TNF-α was cleaved by tumor necrosis factor-alpha-converting enzyme (TACE); however, a point mutation was caused to occur on DNA of TNF-α so that alanine-valine (A-V), a site recognized by TACE in the amino acid sequence of TNF-α, became proline-valine (P-V), thereby allowing TNF-α to remain attached to a cell membrane. The point mutation was performed, in human mTNF-α gene represented by SEQ ID NO: 7, by replacing guanine, which is the 226^{th} base, with cytosine, and replacing adenine, which is the 228^{th} base, with guanine.

Primers suitable for each gene for transduction were used to amplify, through PCR, the coding sequence (CDS) of the transgene (Table 1).

TABLE 1

| | | Sequence information | SEQ ID |
|---|---|---|---|
| | Primer | (5′->3′) | NO |
| 4- | 4-1BBL | TCTAGAGCTAGCGAATTCGCCAC | SEQ ID |
| 1BBL | Forward | CATGGAATACGCCTCTGACGCTT | NO: 10 |
| | 4-1BBL | TTCGCGGCCGCGGATCCTTATTC | SEQ ID |
| | Reverse | CGACCTCGGTGAAGG | NO: 11 |
| mbIL- | mbIL-21 | TAGAGCTAGCGAATTCGCCACCG | SEQ ID |
| 21 | Forward | CCACCATGGCTCTGCCC | NO: 12 |
| | mbIL-21 | TCGCGGCCGCGGATCCTCAATAC | SEQ ID |
| | Reverse | AGGGTGATGACC | NO: 13 |
| OX40L | OX40L | TAGAGCTAGCGAATTCGCCACCA | SEQ ID |
| | Forward | TGGAACGGGTGCAAC | NO: 14 |
| | OX40L | TCGCGGCCGCGGATCCTCACAAG | SEQ ID |
| | Reverse | ACACAGAACTCCCC | NO: 15 |
| mTNF- | mTNF-α | TAGAGCTAGCGAATTCGCCACCG | SEQ ID |
| α | Forward | CCACCATGGCTCTGCCC | NO: 16 |
| | mTNF-α | TCGCGGCCGCGGATCCTCACAGG | SEQ ID |
| | Reverse | GCAATGATCCC | NO: 17 |

Table 1 shows the primers used in the experiment.

The transgene and the lentiviral vector were treated with EcoRI and BamHI restriction enzymes. Thereafter, ligation was performed using an In-Fusion HD cloning kit (Clontech, 639649). The ligated lentiviral vector was transformed into DH5α competent cells and culture was performed. Plasmid DNA was obtained from the transformed DH5α competent cells using a plasmid mini-prep kit (MACHEREY-NAGEL/740422.50). For all plasmid DNA, a request for sequencing thereof was made to an outside company, and it was confirmed that DNA sequences thereof were accurate.

### Example 1.2. Construction of Enriched Lentiviruses

For recombinant lentiviral production, 293T cell line was inoculated into a 75T flask (Nunc, 156499) at 1.5×10⁶ to 2×10⁶ cells two days prior to transfection, and incubated in an incubator at a condition of 5% CO₂ and 37° C. When cell confluency of the 293T cells reached about 80% to 90%, the medium was replaced with 6 ml of OPTI-MEM (Gibco, 31985-088) and incubated for 30 minutes under a 5% CO₂ condition at a temperature of 37° C. A DNA mixed solution and a lipofectamine (lipofectamine 2000, Life technologies, 11668500) mixed solution were prepared (Table 2).

TABLE 2

| Category | Ingredient |
|---|---|
| DNA mixed solution | 6 μg of target DNA, 6 μg of Gag, 6 μg of |
| | REV, 3 μg of VSVG, 1 ml of OPTI-MEM |
| Lipofectamine mixed | 36 μl of lipofectamine 2000, 1 ml of OPTI- |
| solution | MEM |

Table 2 shows the DNA mixed solution and the lipofectamine (lipofectamine 2000, Life technologies, 11668500) mixed solution.

Ingredients of the respective mixed solutions were mixed well using a vortexer and allowed to stand at room temperature for 3 minutes. Then, the two mixed solutions were mixed and allowed to stand at room temperature for 20 minutes or longer. The 293T cells for which medium replacement had been made were subjected to treatment with 2 ml of the mixed solution of DNA and lipofectamine. After 4 hours, the medium was replaced with DMEM (Gibco, 11995073) medium supplemented with 10% (v/v) FBS, and incubation was performed for 48 hours under a condition of 5% CO₂ at a temperature of 37° C.

8 ml of culture of the 293T cells obtained by performing incubation for 48 hours was collected and filtered with a 0.45 μm filter (Millipore, SLHP033RS). The filtered culture was enriched to 250 μl or less using the Amicon Ultra-15 Centrifugal Filter Unit with Ultracel-100 membrane (Merckmillipore, UFC910096). The enriched viruses were aliquoted in appropriate amounts and stored at a temperature of −80° C.

### Example 2. Construction of T cells Having Transduced Genes

### Example 2.1. Lentiviral Infection

0.5×10⁶ cells of T cell line in culture, 1 ml of OPTI-MEM, 50 μl of lentiviral thaw, 10 μg/ml of polybrene (Santa Cruz, C2013) were mixed. The mixture was placed in a 6-well plate (Nunc, 140675), and spinoculation was performed at 1800 g and a temperature of 32° C. for 90 minutes. Then, incubation was performed for 2 hours in an incubator at a condition of 5% CO₂ and 37° C. Thereafter, the medium was replaced with the same culture medium and incubation was performed for 48 hours.

Hut78 cell line (ATCC, TIB-161™) was cultured in IMDM (ATCC, 30-2005) medium containing 20% (v/v) FBS. In subculture, the cell concentration thereof was maintained at 1.5×10⁵ cells/ml to 2.0×10⁵ cells/ml. H9 cell line (ATCC, HTB-176™) and Jurkat T cell line (ATCC, TIB-152™) were cultured in RPMI1640 (ATCC, 30-2001) medium containing 10% (v/v) FBS. In subculture, the cell concentrations thereof were maintained at 1.0×10⁵ cells/ml to 1.5×10⁵ cells/ml and 0.5×10⁵ cells/ml to 1.0×10⁵ cells/ml, respectively. Subculture of all cell lines was performed at 2-day to 3-day intervals. As a culture vessel, a 75T flask was used, and the medium volume was maintained at 15 ml to 20 ml.

The recombinant lentivirus-infected cell line was selected using antibiotics (Table 3).

TABLE 3

| Combination of | | |
|---|---|---|
| transduced genes | Cell line | Antibiotic concentration used |
| mTNF-α | Hut78 | 1 μg/ml of puromycin |
| mbIL-21 | H9 | (Life technologies, A1113802) |
| | Jurkat | or 6 μg/ml of blasticidin (Gibco, R210-01) |
| OX40L | Hut78 | 1 μg/ml of puromycin |
| 4-1BBL | H9 | or 50 μg/ml of G418 (Sigma Aldrich, G8168) |
| | Jurkat | 1 mg/ml of G418 |
| mTNF-α/OX40L | Hut78 | 1 μg/ml of puromycin or 6 μg/ml of blasticidin |
| mbIL-21/OX40L | H9 | 50 μg/ml of G418 |
| mbIL-21/4-1BBL | Jurkat | 0.5 μg/ml of puromycin |
| | | 1 mg/ml of G418 |
| mTNF-α/mbIL-21/4-1BBL | Hut78 | 1 μg/ml of puromycin |
| mTNF-α/mbIL-21/OX40L/4-1BBL | | 6 μg/ml of blasticidin |
| | | 50 μg/ml of G418 |

Table 3 shows antibiotics used for the cell lines having transduced genes.

### Example 2.2. Identification of Expression of Transduced Genes

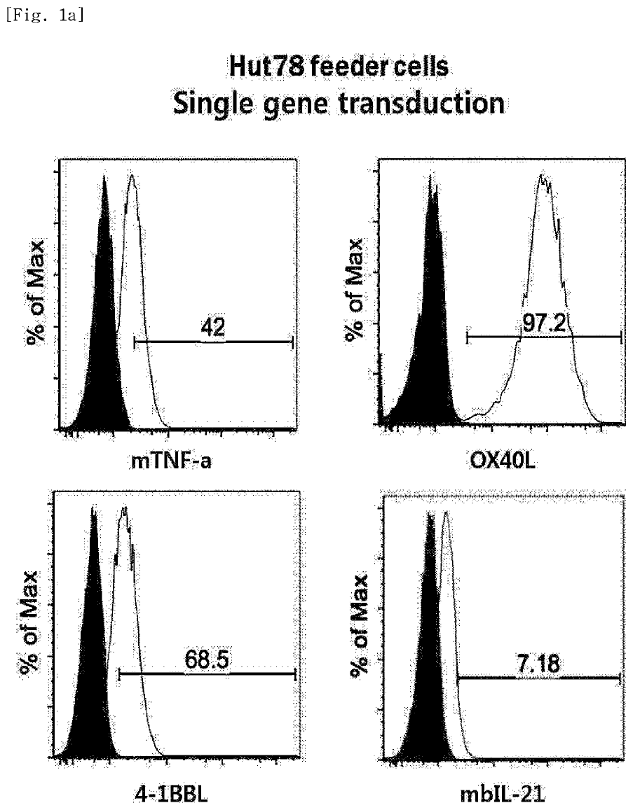

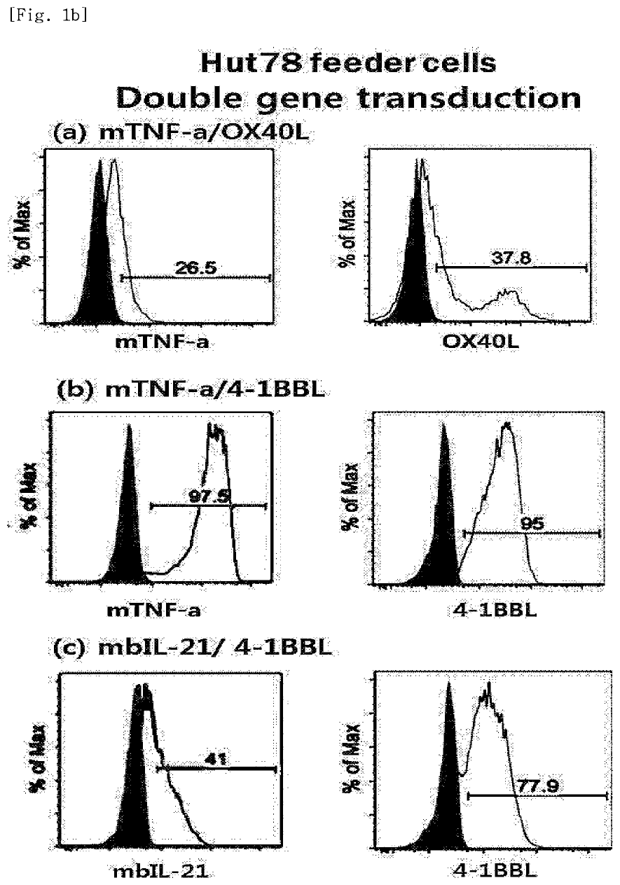

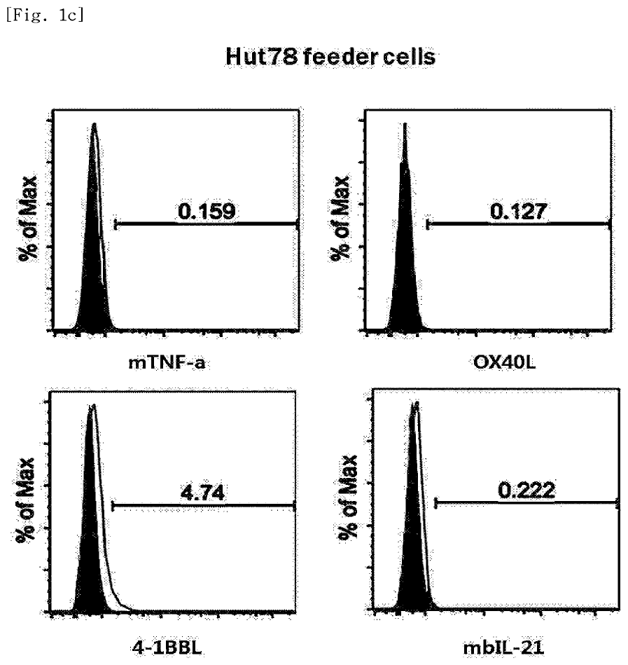

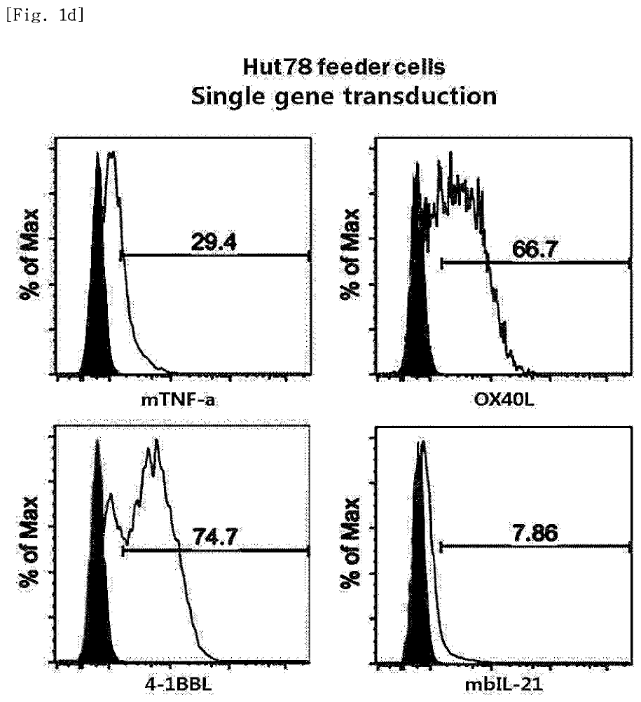

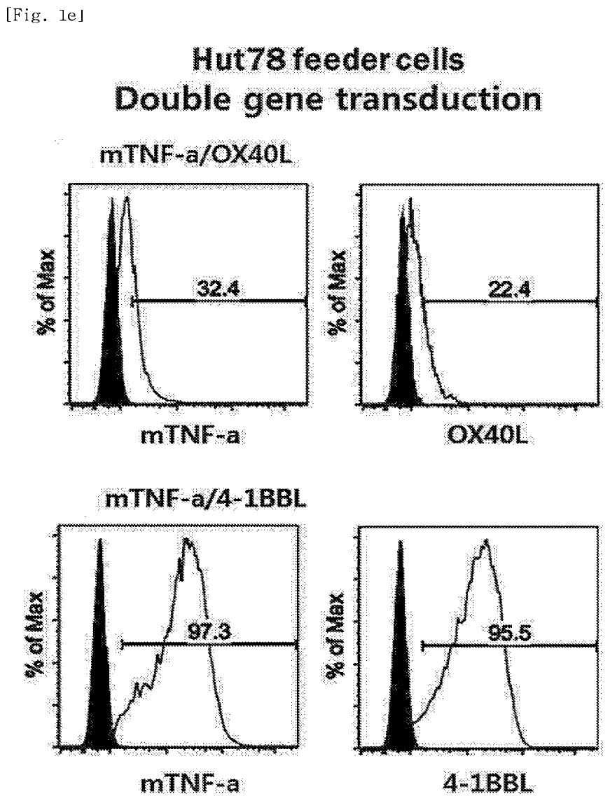

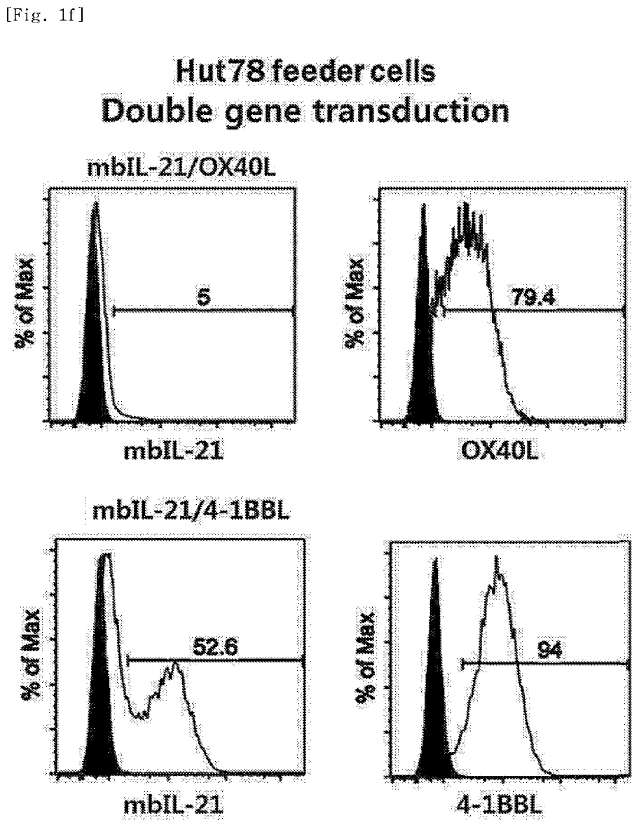

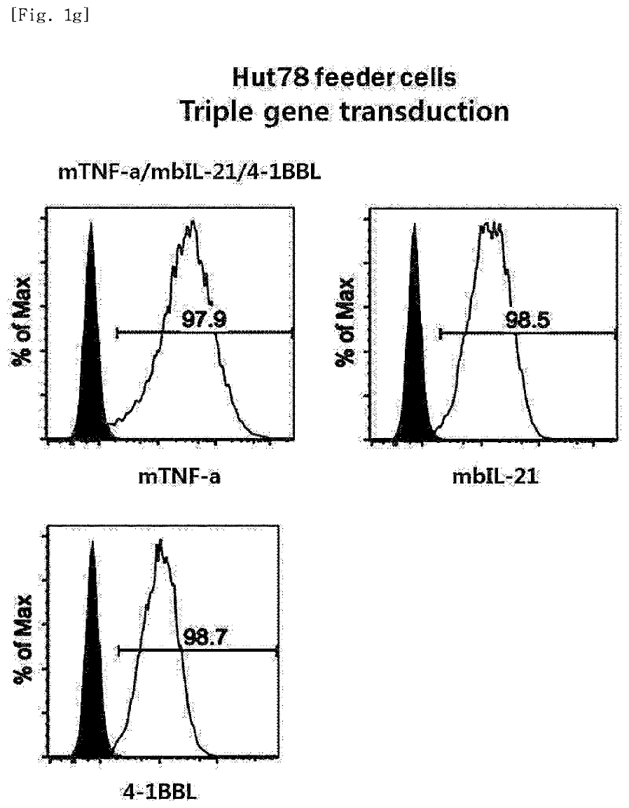

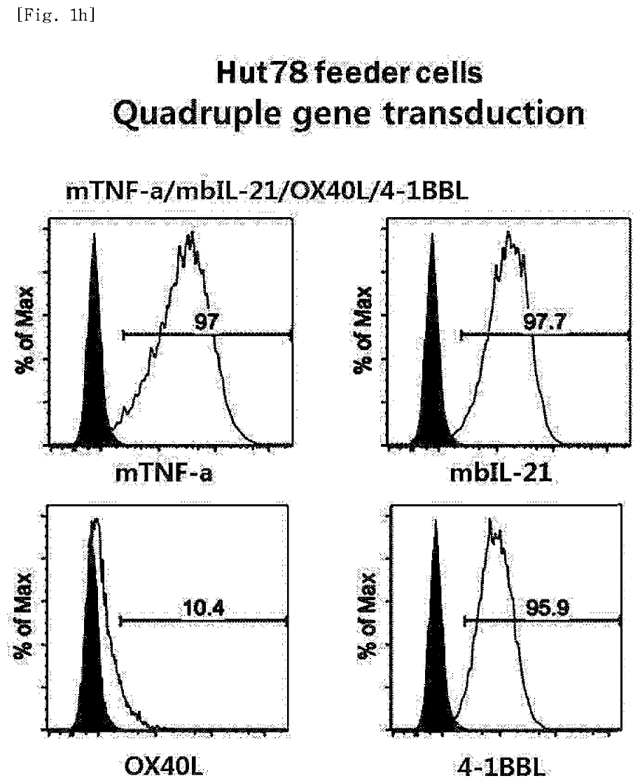

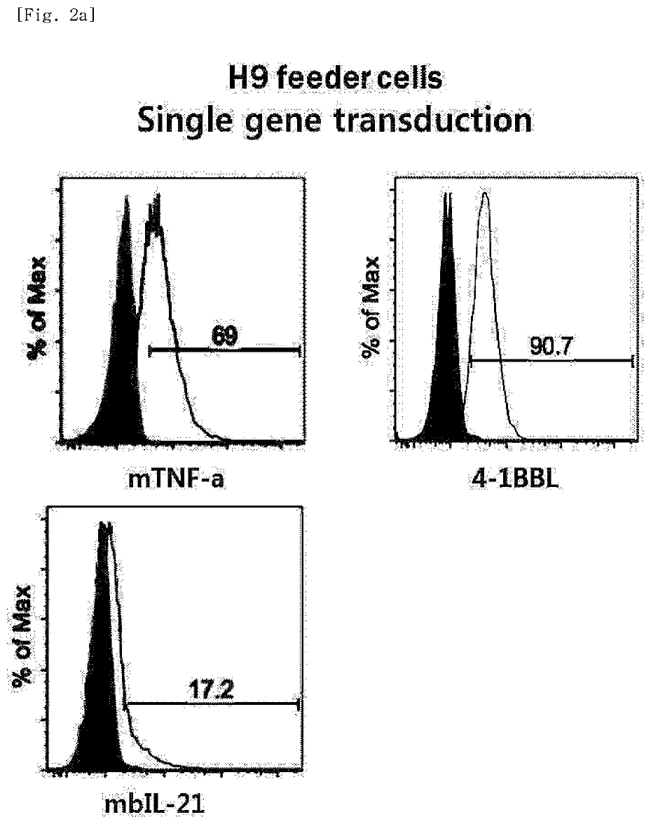

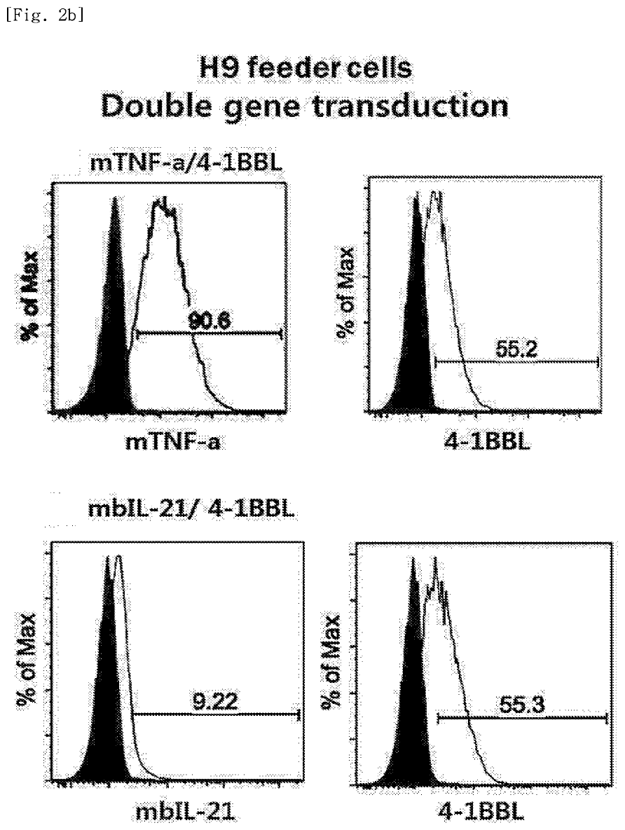

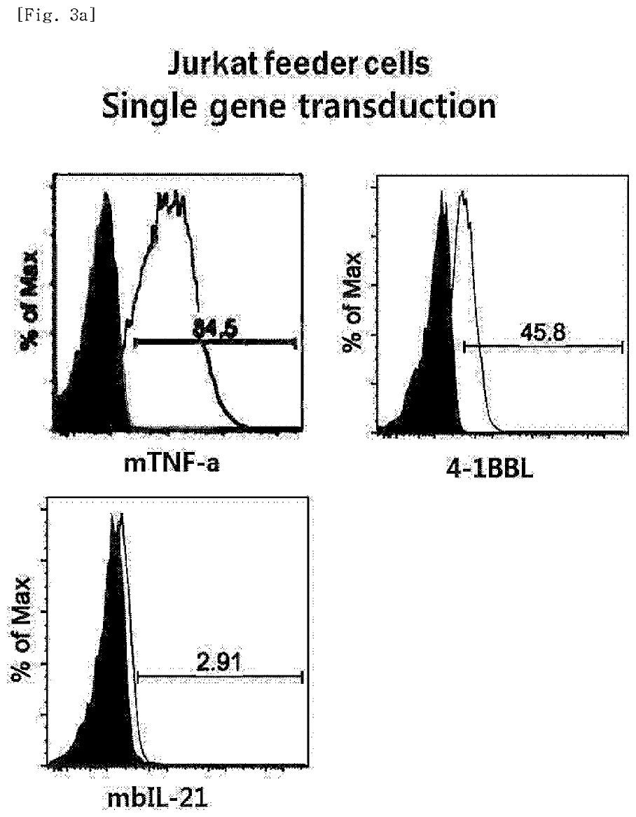

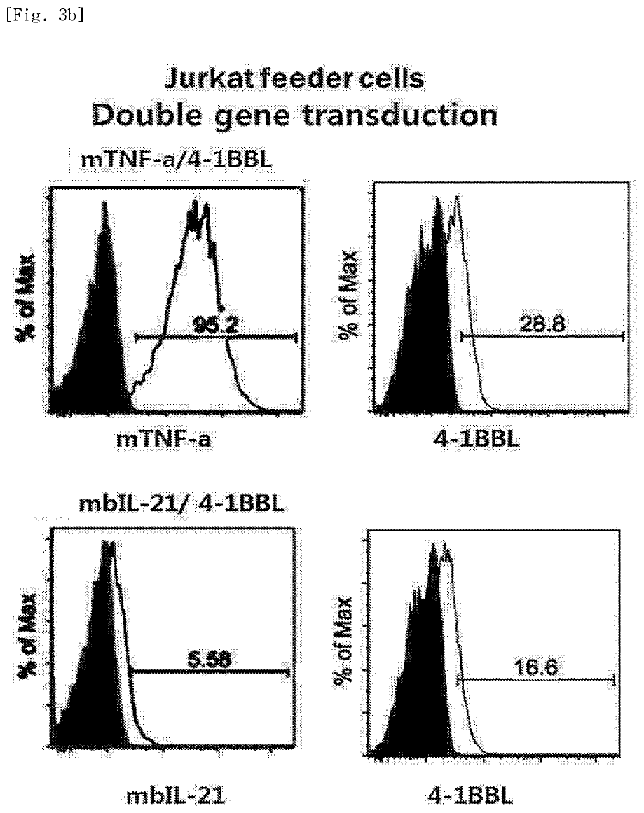

The T cell line subcultured in Example 2.1. was collected and centrifuged at 1,200 rpm for 5 minutes. Then, the culture was removed by suction. FACS buffer was made by adding 2% (v/v) FBS to PBS. Dilution with 1 ml of the FACS buffer was performed and the cell number was measured. Dilution with the FACS buffer was performed so as to give a concentration of 5×10⁶ cells/ml. 100 μl of the diluted cell solution was added to each 5 ml FACS tube (Falcon, 352052). Staining was performed with anti-human TNF-α(membrane)-PE (R&D Systems, FAB210P), anti-human OX40L-PE (BD, 558184), anti-human 4-1BBL-PE (BD, 559446), anti-human IL-21-PE (eBioscience, 12-7219-42), 7-AAD (Beckman Coulter, Inc., IM3630c), PE mouse IgG1 k isotype control (BD Pharmingen, 555749), PerCP-Cy5.5 mouse IgG1 k isotype control (BD, 550795) antibodies, and then an expression rate of each gene was analyzed using FACS equipment (FIGS. 1a to 3b).

### Example 3. Co-Culture of CD3(−) PBMCs and T cells Having Transduced Genes

### Example 3.1. Preparation of CD3(−) PBMC Seed Cells

Phosphate buffered saline (PBS, LONZA, 17-516Q) was added, at a 1:1 ratio, to PBMCs collected from healthy donors and centrifugation was performed at 1,500 rpm and a temperature of 4° C. for 10 minutes. 2% (v/v) FBS and 2 mM EDTA were added to PBS, to make MACS running buffer. The PBMC pellets were suspended in 10 ml of the MACS running buffer and the cell number was measured using an Adam cell counter system.

In order to obtain the seed cells from which CD3(+) cells have been eliminated, 5×10⁷ PBMCs were transferred to a new 50-ml tube, and then centrifuged at 1,200 rpm and a temperature of 4° C. for 10 minutes. 400 μl of the MACS running buffer and 100 μl of CD3 magnetic beads (Miltenyi Biotech, 130050101) were added to 5×10⁷ PBMC cell pellets and reaction was allowed to occur at a temperature of 4° C. for 20 minutes. 10 ml to 20 ml of the MACS running buffer was added thereto and washing was performed. Then, centrifugation was performed at 1,200 rpm and a temperature of 4° C. for 10 minutes, and the resultant was suspended again in 2 ml of the MACS running buffer.

The cells were isolated using VarioMACS (Miltenyi Biotech) equipped with a CS column (Miltenyi Biotech, 130-041-305). The cells were recovered by washing the column until it reached a final volume of 20 ml. The cell number was measured using the Adam cell counter system. 1×10⁷ cells were placed in a new 50-ml tube, and centrifuged at 1,200 rpm and a temperature of 4° C. for 10 minutes. The cell pellets were suspended in freezing medium, and freezing was performed in liquid nitrogen to achieve 1×10⁷ cells per vial.

One frozen CD3(−) PBMC vial was thawed and transferred to a 50-ml tube. CD3(−) PBMCs were suspended in PBS containing 0.6% (v/v) ACD (citrate-dextrose solution, Sigma-Aldrich, C3821), 0.2% (v/v) fetal serum bovine (FBS), and 2 mM EDTA, and centrifuged at 1,200 rpm and a temperature of 4° C. for 10 minutes. CD3(−) PBMC pellets were suspended in 1% (v/v) CellGro medium (Cellgenix, 20802-0500), and the cell number was measured using the Adam cell counter system. CD3(−) PBMC seed cells were suspended in 1% (v/v) CellGro medium at a concentration of 1×10⁶ cells/ml.

### Example 3.2. Co-Culture of CD3(−) PBMC Seed Cells and T Feeder Cells Having Transduced Genes

T cells having transduced gene were recovered from the culture flask and centrifuged at 1,200 rpm and a temperature of 4° C. for 10 minutes. Thereafter, the cells were suspended in 1% (v/v) CellGro medium, and the cell number was measured using the Adam cell counter system. T feeder cells having transduced gene(s) were prepared by being suspended in 1% (v/v) CellGro medium at a concentration of 5×10⁶ cells/ml and then being irradiated with 20,000 cGy in a gamma-ray irradiator and inactivated.

In culture of natural killer cells, 500 IU of IL-2 (Proleukin Inj., Novartis Korea) and 10 ng/ml of OKT-3 (eBioscience, 16-0037-85) were added to a culture plastic plate. On day 0 of culture, the CD3(−) PBMC seed cells and the T feeder cells having transduced gene(s) were added in a volume of 0.5 ml to 1 ml each at a ratio of 1:5 or 1:2.5, and CellGro medium containing 1% (v/v) human plasma was added thereto in a volume of 0.25 ml to 0.5 ml. The resultant was subjected to static culture in an incubator at a temperature condition of 37° C. for 4 to 7 days while adding the medium.

On days 4 to 5 of culture, the cell number was measured so that the cells had a concentration of about 0.5×10⁶ to 1×10⁶ cells/ml. Thereafter, the cells were diluted with CellGro medium containing 500 IU of IL-2 and 1% (v/v) autologous plasma, and placed in an appropriate culture vessel. The cells were again subjected to static culture.

After that, the cell number was measured at 2-day to 3-day intervals so that the cells had a concentration of 0.5×10⁵ to 1×10⁶ cells/ml. Thereafter, the cells were subjected to suspension culture until day 21 while performing dilution with CellGro medium containing 500 IU of IL-2 and 1% (v/v) autologous plasma. On day 21 of suspension culture, natural killer cells were obtained.

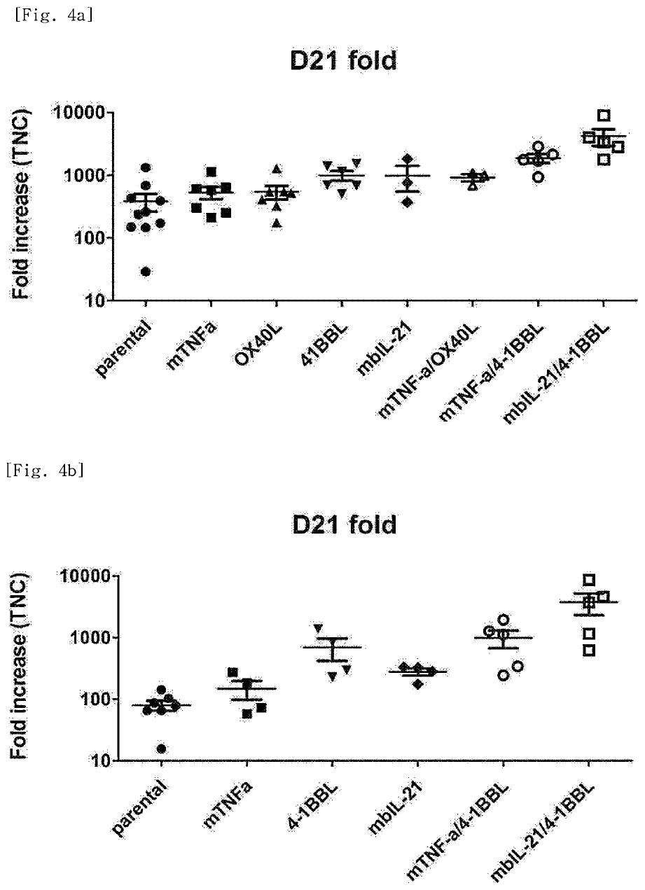

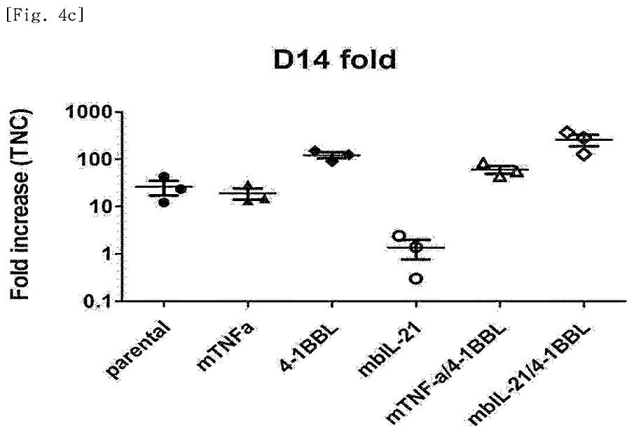

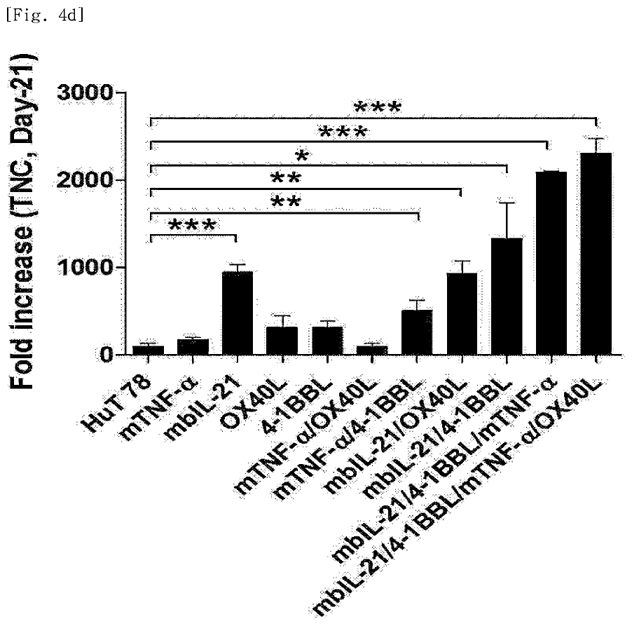

As a result of comparing proliferation rates of the cultured natural killer cells, it was possible to identify that culture with the cell line into which one or more genes (mTNF-α, OX40L, 4-1BBL, or mbIL-21) have been transduced results in increased proliferation of natural killer cells, in terms of proliferation rate based on total nucleated cells (TNC), as compared with the cell line having no transduced gene (76-fold in H9, 382-fold in Hut78, 26-fold in Jurkat). In particular, as compared with single transduction of the 4-1BBL gene or the mbIL-21 gene, in case of the cell line into which the mbIL-21/4-1BBL gene is transduced, remarkably high proliferation rates for natural killer cells, which are 4,169-fold in Hut78-mbIL-21/4-1BBL, 3,758-fold in H9-mbIL-21/4-1BBL, 258-fold in Jurkat-mbIL-21/4-1BBL, were induced regardless of types of T cells (Table 4, FIGS. 4a to 4c). That is, it was identified that a synergistic effect was observed when the mbIL-21 gene and the 4-1BBL gene were transduced.

TABLE 4

| | Hut78 feeder cell | H9 feeder cell | Jurkat feeder cell |
|---|---|---|---|
| Transduced | Fold | Fold | Fold |
| gene(s) | increase (D21) | increase (D21) | increase (D14) |
| Parental | 382.0 ± 379.78 | 79.3 ± 38.60 | 26.1 ± 15.45 |
| mTNF-α | 532.5 ± 315.48 | 147.7 ± 101.04 | 19.1 ± 8.84 |
| OX40L | 541.4 ± 353.22 | — | — |
| 4-1BBL | 992.3 ± 423.75 | 689.8 ± 541.60 | 121.7 ± 30.46 |
| mbIL-21 | 979.2 ± 745.36 | 277.8 ± 71.00 | 1.4 ± 1.05 |
| mTNF-α/OX40L | 916.0 ± 204.52 | — | — |
| mTNF-α/4-1BBL | 1871.7 ± 701.68 | 985.3 ± 705.35 | 60.7 ± 20.09 |
| mbIL-21/4-1BBL | 4169.5 ± 2795.10 | 3758.5 ± 3232.39 | 258.5 ± 121.36 |

Table 4 shows proliferation rates of the natural killer cells cultured with T cell line having undergone single or double gene transduction.

In order to identify a synergistic effect obtained when a combination of two or more genes is transduced, a proliferative effect of the natural killer cells cultured with Hut78 T cell line having undergone triple gene (mTNF-α/mbIL-21/4-1BBL) or quadruple gene (mTNF-α/mbIL-21/OX40L/4-1BBL) transduction was compared together with combinations of genes for which experiments were previously made. Among the cell lines having undergone double gene transduction, the cell line that induced the highest proliferation of natural killer cells was the cell line (998-fold) into which the mbIL-21/4-1BBL gene had been transduced. However, when cultured with a cell line having undergone triple gene transduction (1,607-fold) or a cell line having undergone quadruple gene transduction (1,640-fold), it was identified that a higher synergistic effect was observed than the cell line (998-fold) into which the mbIL-21/4-1BBL gene was transduced (Table 5 and FIG. 4d).

TABLE 5

| | Hut78 feeder cell |
|---|---|
| Transduced gene(s) | Fold increase(D21) |
| Parental | 72.1 ± 71.5 |
| mTNF-α | 121.9 ± 103.0 |
| OX40L | 212.4 ± 297.2 |
| 4-1BBL | 235.1 ± 195.1 |
| mbIL-21 | 737.2 ± 393.6 |
| mTNF-α/OX40L | 77.8 ± 67.3 |
| mTNF-α/4-1BBL | 385.4 ± 293.3 |
| mbIL-21/4-1BBL | 998.3 ± 911.2 |
| mbIL-21/OX40L | 743.0 ± 411.7 |
| mTNF-α/mbIL-21/4-1BBL | 1607.3 ± 845.0 |
| mTNF-α/mbIL- | 1640.6 ± 1191.6 |
| 21/OX40L/4-1BBL | |
| Single culture of seed cells | 0.8 |

Table 5 shows proliferation rates of the natural killer cells cultured with T cell lines having undergone multiple gene transduction.

Experiments were conducted to identify whether culture of natural killer cells can be continuously maintained with repeated re-stimulation by T cell line due to gene transduction.

Hut78 cells having undergone triple gene transduction were recovered from the culture flask and centrifuged at 1,200 rpm and a temperature of 4° C. for 10 minutes. Thereafter, the cells were suspended in 1% (v/v) CellGro medium, and the cell number was measured using the Adam cell counter system. Hut78 feeder cells having undergone triple gene transduction were prepared by being suspended in 1% (v/v) CellGro medium at a concentration of 2.5×10⁶ cells/ml and then being irradiated with 20,000 cGy in a gamma-ray irradiator and inactivated.

In culture of natural killer cells, 500 IU of IL-2 (Proleukin Inj., Novartis Korea) and 10 ng/ml of OKT-3 (eBioscience, 16-0037-85) were added to a culture plastic plate.

On day 0 of culture, the CD3(−) PBMC seed cells and the Hut78 feeder cells having undergone triple gene transduction were added in a volume of 0.5 ml to 1 ml each at a ratio of 1:2.5, and CellGro medium containing 1% (v/v) human plasma was added thereto in a volume of 0.5 ml to 1 ml. The cells were subjected to static culture in an incubator at a temperature condition of 37° C. for 4 to 7 days. On day 2 or 3 of culture, half of the medium was removed and CellGro medium containing 1% (v/v) human plasma was added thereto as much as the removed volume. On day 4 or 5 of culture, the cell number was measured so that the cells had a concentration of about 0.5×10⁶ cells/ml to 1×10⁶ cells/ml.

On day 7 or 11 of culture, the cell number was measured and the cells were suspended in 1% (v/v) CellGro medium at a concentration of 1×10⁶ cells/ml. For re-stimulation, the Hut78 feeder cells having undergone triple gene transduction were added thereto in a volume of 0.5 ml to 1 ml each at a ratio of 1:2.5, and CellGro medium containing 500 IU of IL-2 (Proleukin Inj., Novartis Korea), 10 ng/ml of OKT-3 (eBioscience, 16-0037-85), and 1% (v/v) human plasma was added thereto in a volume of 0.5 ml to 1 ml. The cells were subjected to static culture in an incubator at a temperature condition of 37° C. for 3 to 4 days. After that, the cell number was measured at 2-day or 3-day intervals so that the cells had a concentration of 0.5×10⁵ cells/ml to 1×10⁶ cells/ml.

The re-stimulation process was repeated at 7-day or 11-day intervals, so that a total of 4 re-stimulations at 7-day intervals (D7RS5) and a total of 3 re-stimulations at 11-day intervals (D11RS4) were given.

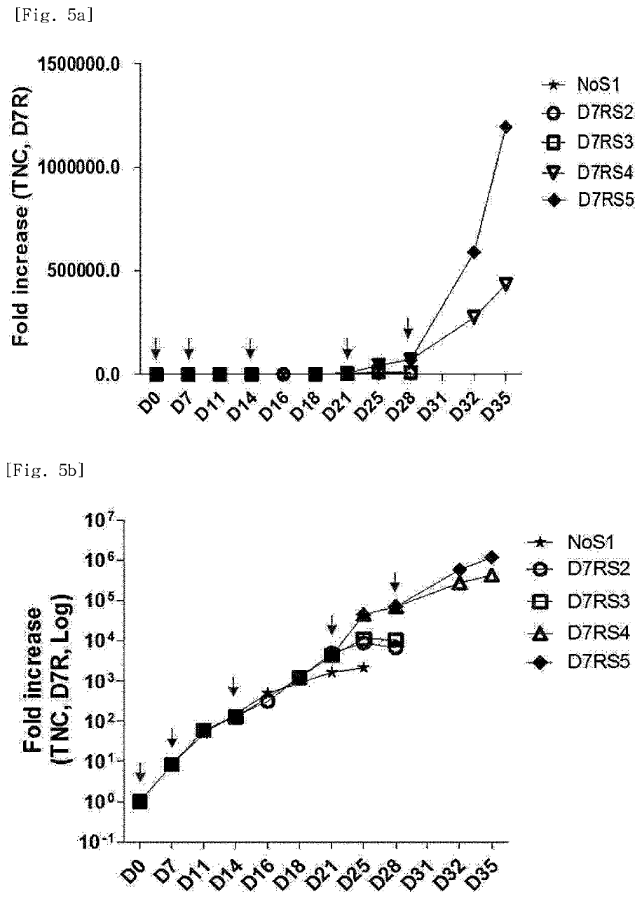

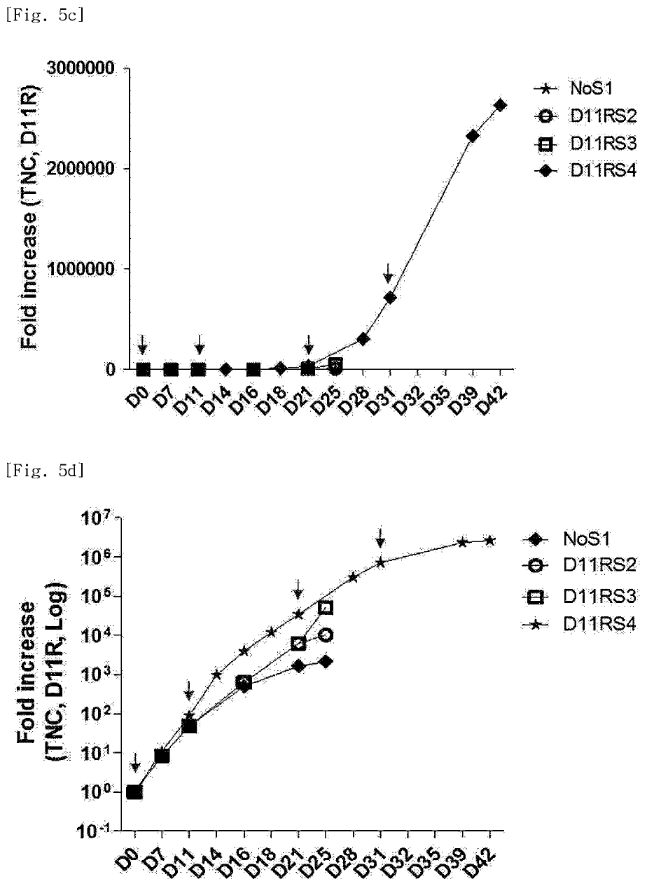

As a result of comparing proliferation rates of cultured natural killer cells, it was possible to identify that culture with one or more re-stimulations resulted in increased proliferation of natural killer cells, in terms of proliferation based on total nucleated cells (TNC), as compared with a proliferation obtained in a case where no re-stimulation was given (FIGS. 5a to 5d). When being stimulated once with Hut78-mTNF-α/4-1BBL/mbIL-21 cell line on day 0 of culture, culture was continued up to day 25 and 2,124-fold proliferation was achieved. When stimulated 5 times with Hut78-mTNF-α/4-1BBL/mbIL-21 cell line at 7-day intervals, culture was continued up to day 35 day and 436,032-fold proliferation was achieved. When stimulated 4 times with Hut78-mTNF-α/4-1BBL/mbIL-21 cell line at 11-day intervals, culture was continued up to day 42 and 2,628,153-fold proliferation was achieved. Therefore, it was possible to identify that repeated stimulation with Hut78-mTNF-α/4-1BBL/mbIL-21 T cell line at 7-day or 11-day intervals resulted in a continuous increase in proliferation of natural killer cells.

### Experimental Example 1. Identification of Cell Viability of Natural Killer Cells Depending on Transduced Gene(s)

In order to compare and evaluate in-vitro cell viability, the Adam cell counter system, which is one of cell counters using PI staining solution capable of binding to intracellular nuclei, was used. After the number of viable cells was calculated by subtracting the number of dead cells from the measured total number of cells, cell viability was calculated using the following Expression I.

### [Expression I]

Cell viability (%)=(Number of viable cells/number of total cells)×100

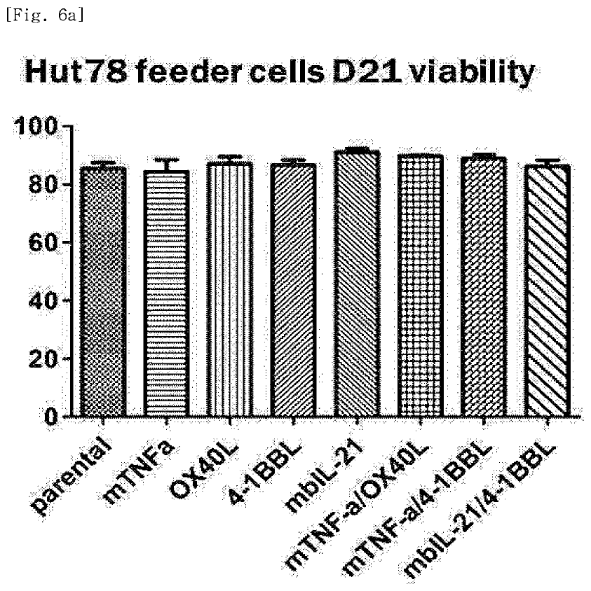

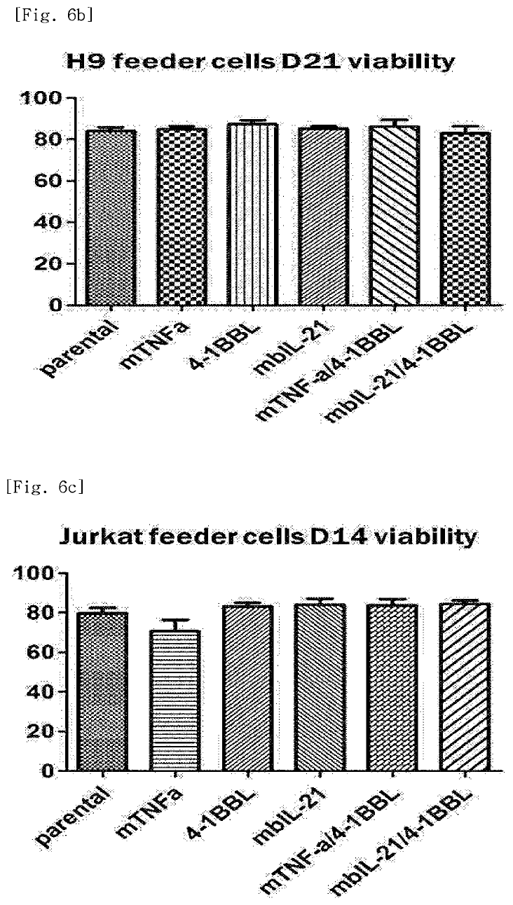

As a result of measuring viability of the natural killer cells after 21 days of culture, high cell viability of about 80% or more was observed under all conditions (FIGS. 6a to 6d).

### Experimental Example 2. Cell Phenotypic Analysis in Natural Killer Cells Depending on Transduced Gene(s)

The natural killer cells cultured for 21 days or the natural killer cells cultured with repeated re-stimulation were collected and centrifuged at 1,200 rpm for 5 minutes. The culture was removed by suction. Dilution with 1 ml of FACS buffer was performed and the cell number was measured. Dilution with the FACS buffer was performed so as to give a concentration of 5×10⁶ cells/ml. 100 μl of the diluted cell solution was added to each 5-ml FACS tube (Falcon, 352052), and phenotypic analysis was performed with the following antibodies.
Tube 1: Anti-human CD3-FITC (BD Pharmingen, 555332), anti-human CD16-PE (BD Pharmingen, 555407), anti-human CD56-PE-Cy5 (BD Pharmingen, 555517) or anti-human CD56-PE Cy7 (BD Pharmingen, 557747), anti-human CD56-BV421 (BD Pharmingen, 562751)
Tube 2: Anti-human CD14-FITC (BD Pharmingen, 555397), anti-human CD19-PE (BD Pharmingen, 555413), anti-human CD3-PE-Cy5 (BD Pharmingen, 555341) or anti-human CD3-PE Cy7 (eBioscience, 25-0038-42), anti-human CD3-BV421 (BD Pharmingen, 562438)
Tube 3: Anti-human CD3-FITC, anti-human NKG2D-PE (R&D Systems, FAB139P), anti-human CD56-PE-Cy5 or anti-human CD56-PE-Cy7, anti-human CD56-BV421
Tube 4: Anti-human CD3-FITC, anti-human NKp30-PE (BD Pharmingen, 558407), anti-human CD56-PE-Cy5 or anti-human CD56-PE-Cy7, anti-human CD56-BV421
Tube 5: Anti-human CD3-FITC, anti-human NKp44-PE (BD Pharmingen, 558563), anti-human CD56-PE-Cy5 or anti-human CD56-PE-Cy7, anti-human CD56-BV421
Tube 6: Anti-human CD3-FITC, anti-human NKp46-PE (BD Pharmingen, 557991), anti-human CD56-PE-Cy5 or anti-human CD56-PE-Cy7, anti-human CD56-BV421
Tube 7: Anti-human CD3-FITC, anti-human DNAM-1-PE (BD Pharmingen, 559789), anti-human CD56-PE-Cy5 or anti-human CD56-PE-Cy7, anti-human CD56-BV421
Tube 8: Anti-human CD3-FITC, anti-human CXCR3-PE (BD Pharmingen, 557185), anti-human CD56-PE-Cy5 or anti-human CD56-PE-Cy7, anti-human CD56-BV421
Tube 9: Anti-human CD3-FITC, PE mouse IgG1 k isotype control (BD Pharmingen, 555749), anti-human CD56-PE-Cy5 or anti-human CD56-PE-Cy7, anti-human CD56-BV421
Tube 10: FITC mouse IgG1 k isotype control (BD Pharmingen, 555748), PE mouse IgG1 k isotype control, PE-Cy5 mouse IgG1 k isotype control (BD Pharmingen, 555750) or PE-Cy7 mouse IgG1 k isotype control (BD Pharmingen, 557872), BV421 mouse IgG1 k isotype control (BD Pharmingen, 562438)
Tube 11: Anti-human CD3-FITC, anti-human NKG2A-PE (R&D Systems, FAB1059P), anti-human CD56-PE-Cy5 or anti-human CD56-PE-Cy7, anti-human CD56-BV421
Tube 12: Anti-human CD3-FITC, anti-human NKG2C-PE (R&D Systems, FAB138P), anti-human CD56-PE-Cy5 or anti-human CD56-PE-Cy7, anti-human CD56-BV421
Tube 13: Anti-human CD3-FITC, anti-human CD62L-PE (eBioscience, 12-0629-42), anti-human CD56-PE-Cy5 or anti-human CD56-PE-Cy7, anti-human CD56-BV421
Tube 14: Anti-human CD3-FITC, anti-human CD69-PE (R&D Systems, FAB23591P), anti-human CD56-PE-Cy5 or anti-human CD56-PE-Cy7, anti-human CD56-BV421
Tube 15: Anti-human CD3-FITC, anti-human CD57-PE (BD Pharmingen, 560844), anti-human CD56-PE-Cy5 or anti-human CD56-PE-Cy7, anti-human CD56-BV421

In Tube 1, Staining with anti-human CD56 was performed by selecting one of three fluorescences, and thus the same fluorescence was selected for CD3 of Tube 2 and CD56 of Tubes 3 to 9 and 11 to 15, and isotype control of Tube 10.

The cells in the above tubes were stained at refrigeration temperature for 30 minutes. Thereafter, 2 ml of FACS buffer was added to the stained cells, and centrifugation was performed at 1,500 rpm for 5 minutes. The supernatant was removed, and 2 ml of FACS buffer was added again. Centrifugation was performed at 1,500 rpm for 5 minutes. The supernatant was removed again, and placed and suspended in 200 μl of fixation buffer (BD, 554655). Then, identification of the cells and examination for purity and phenotype thereof were performed using FACS LSRII Fortessa (BD).

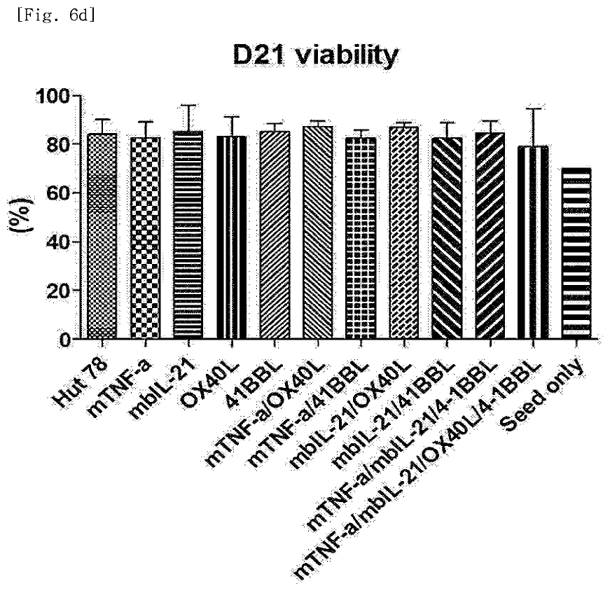

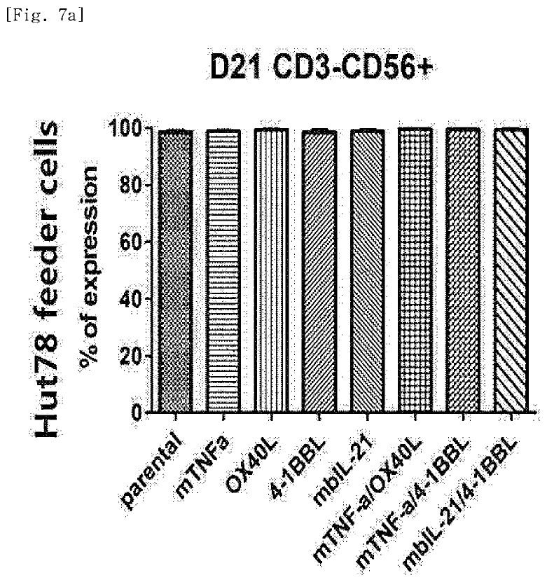

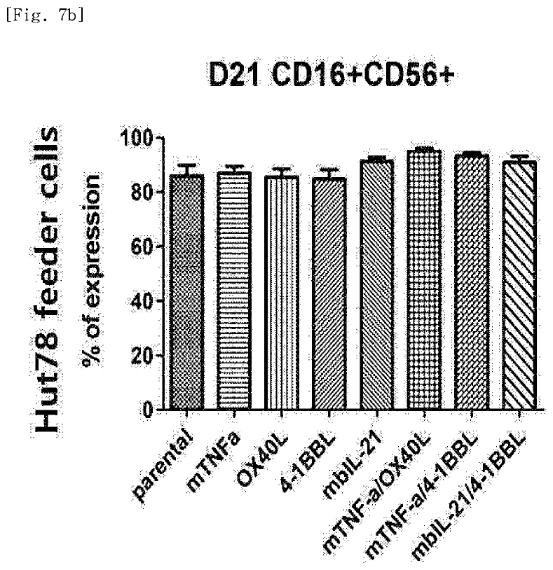

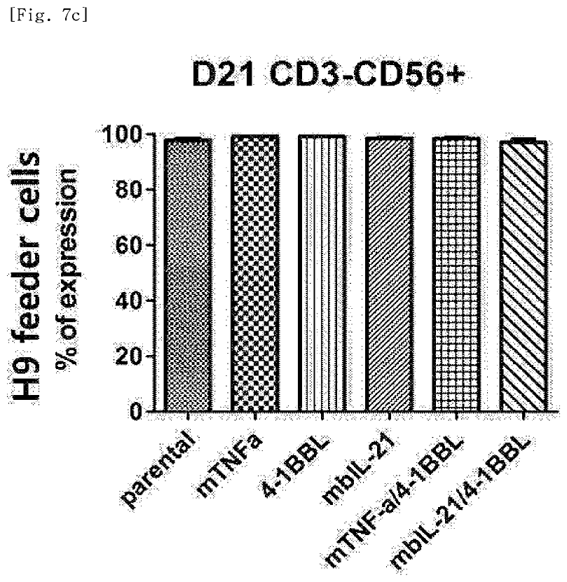

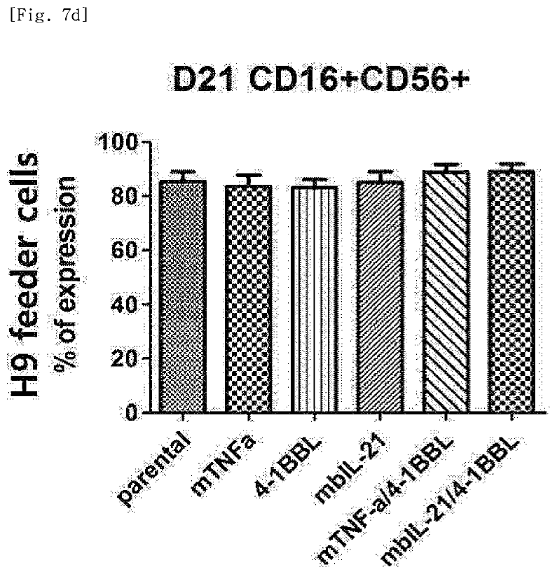

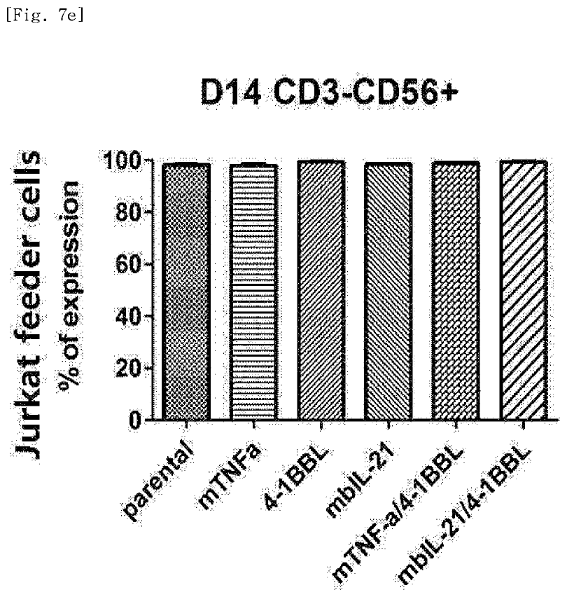

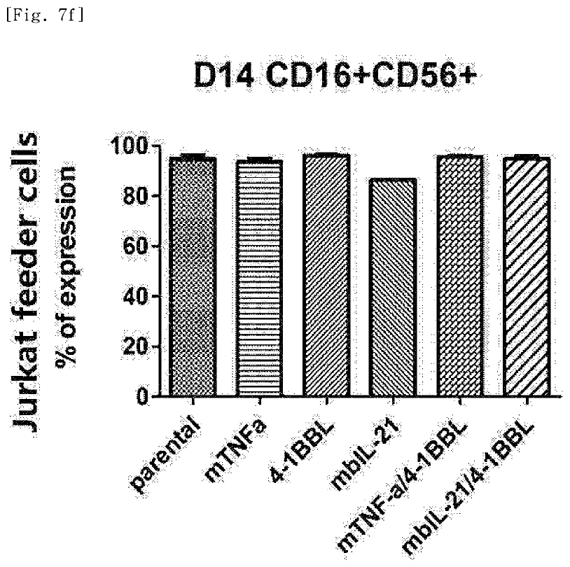

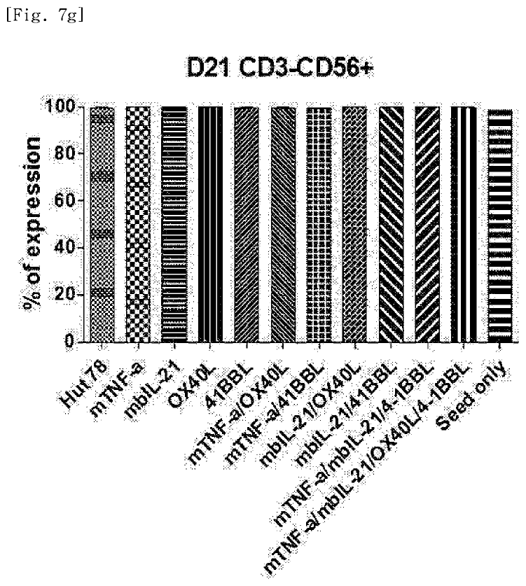

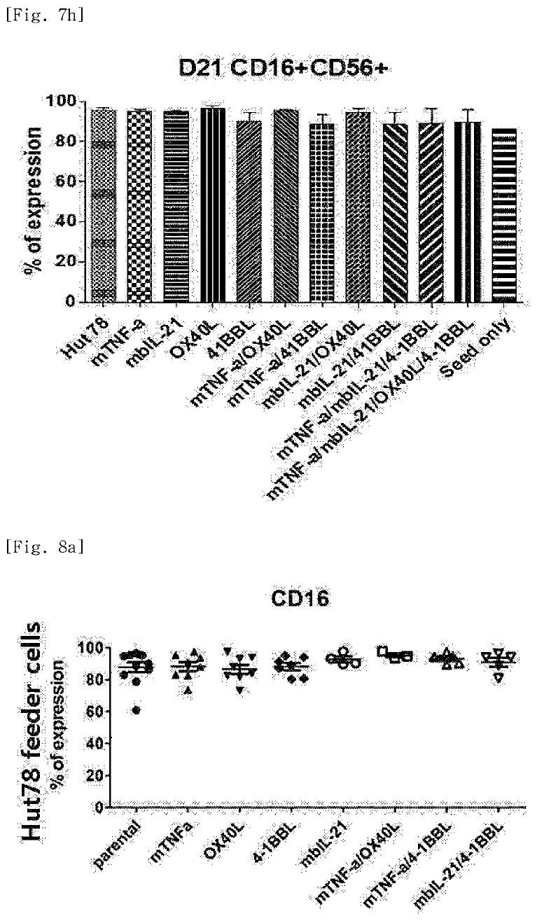

Identification and purity analysis were performed on the natural killer cells cultured for 21 days. As a result, it was identified that the NK cell (CD3-CD56+) content was 95% or more under all conditions, and that an expression level of CD56+CD16+marker for evaluating activation of the natural killer cells was highly maintained using the T feeder cells having undergone double gene transduction (FIGS. 7a to 7h).

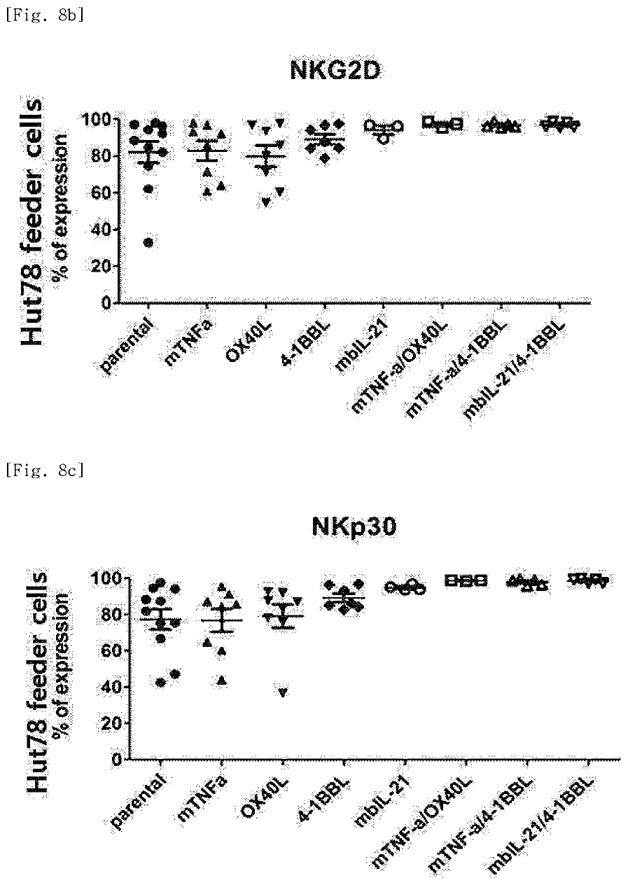

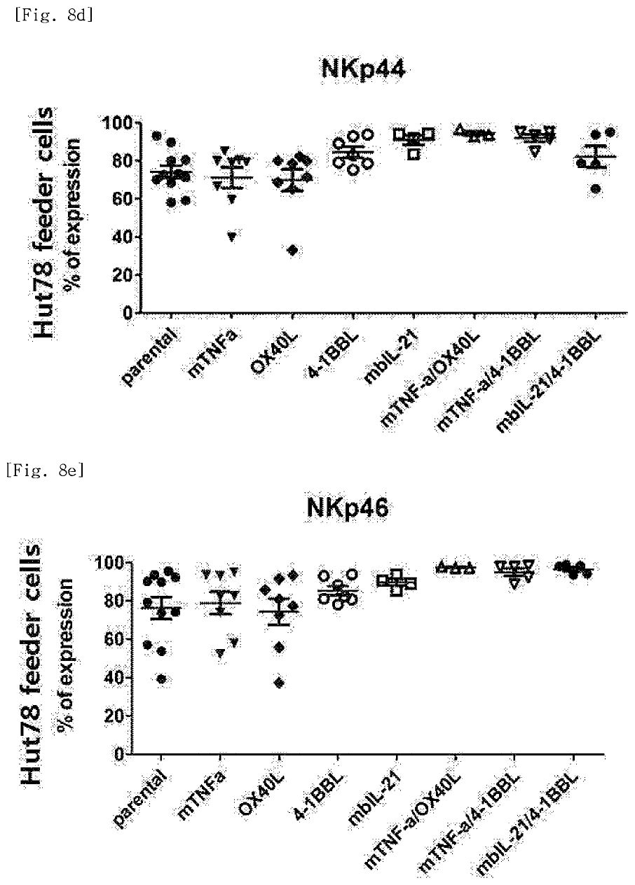

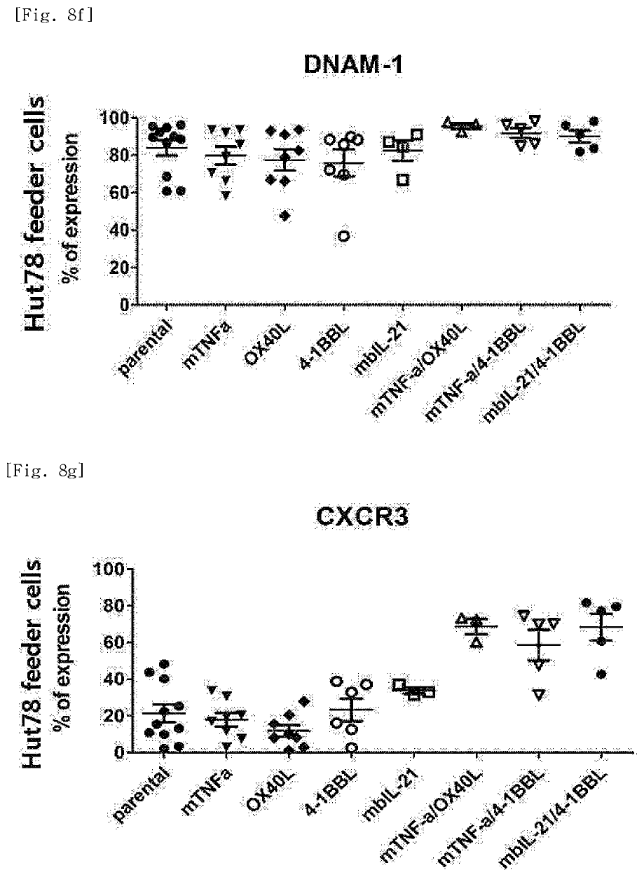

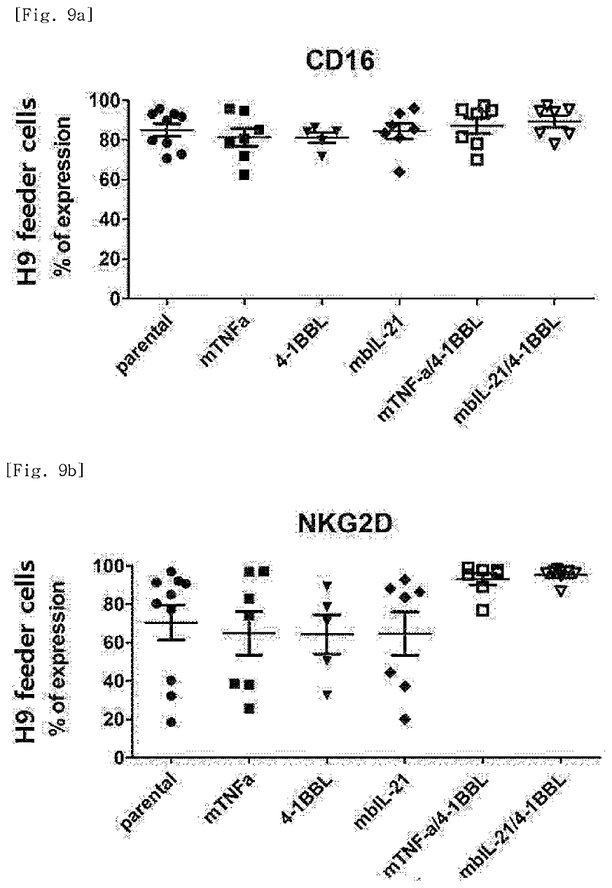

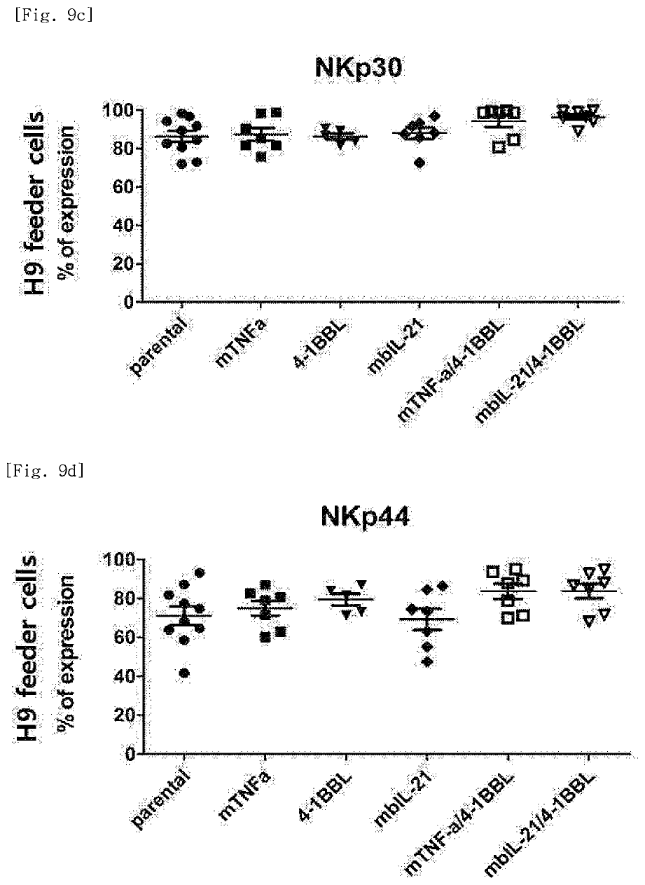

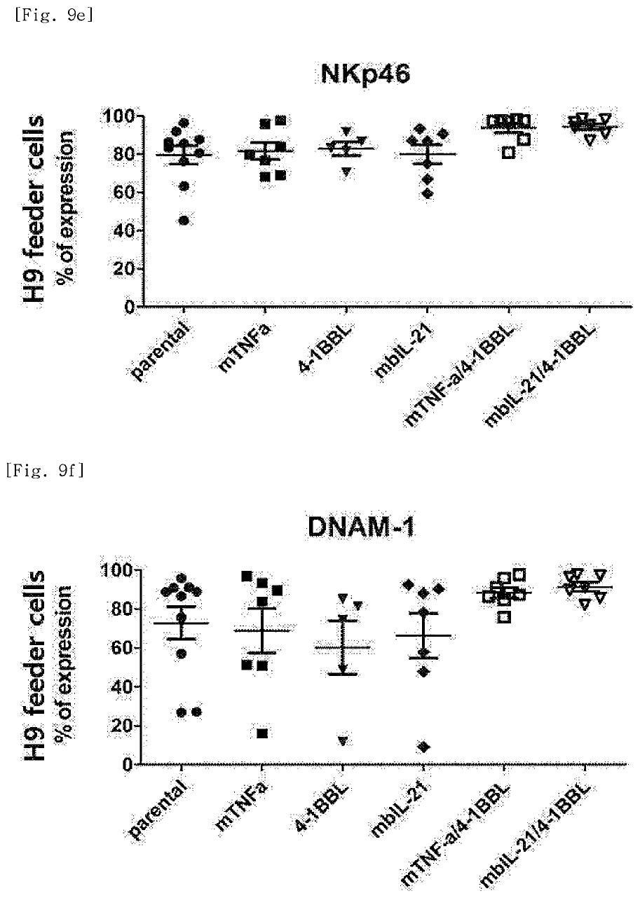

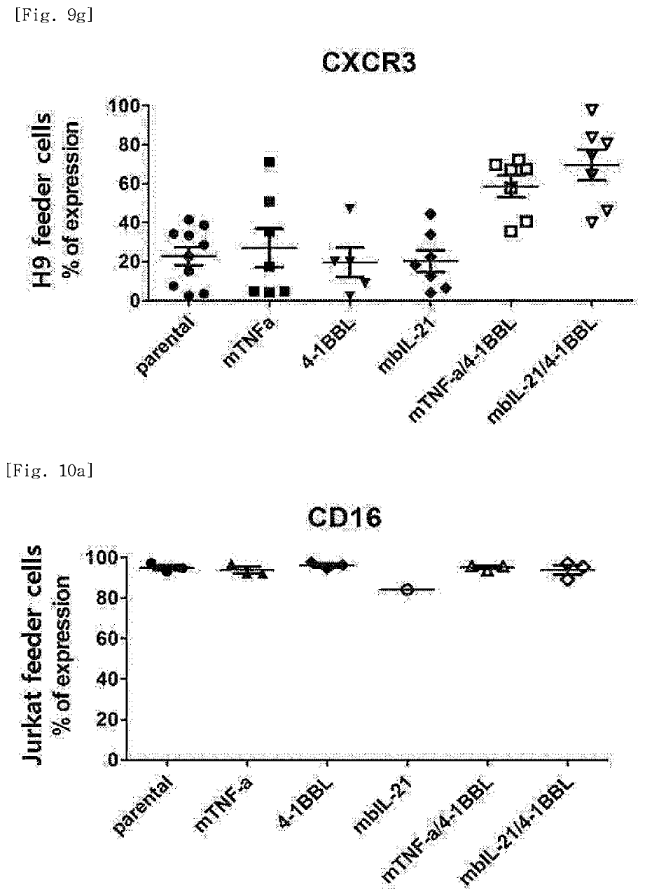

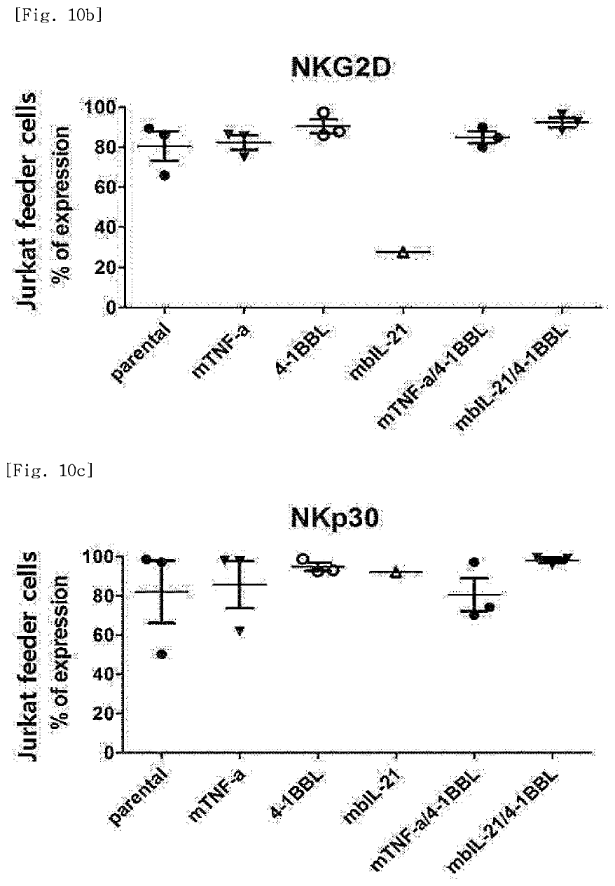

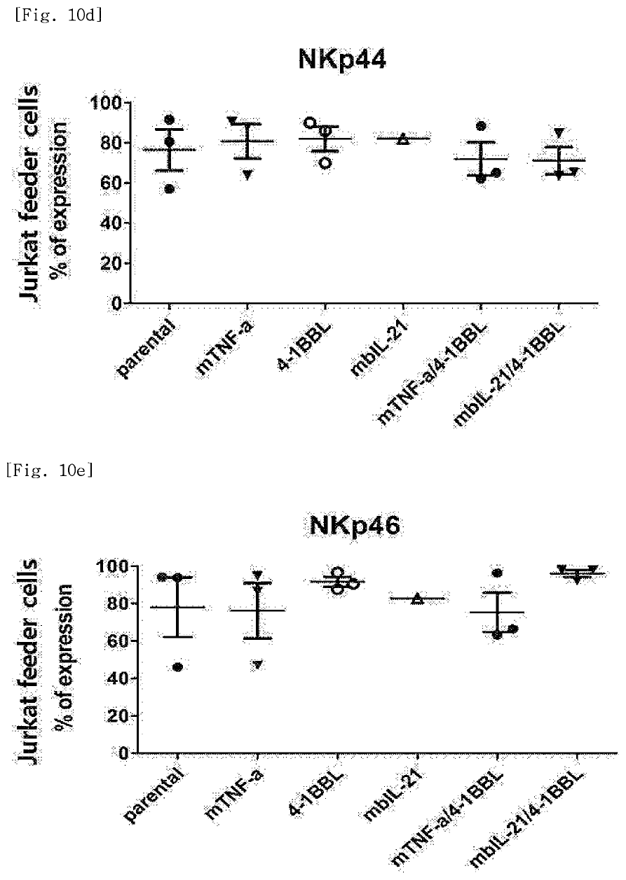

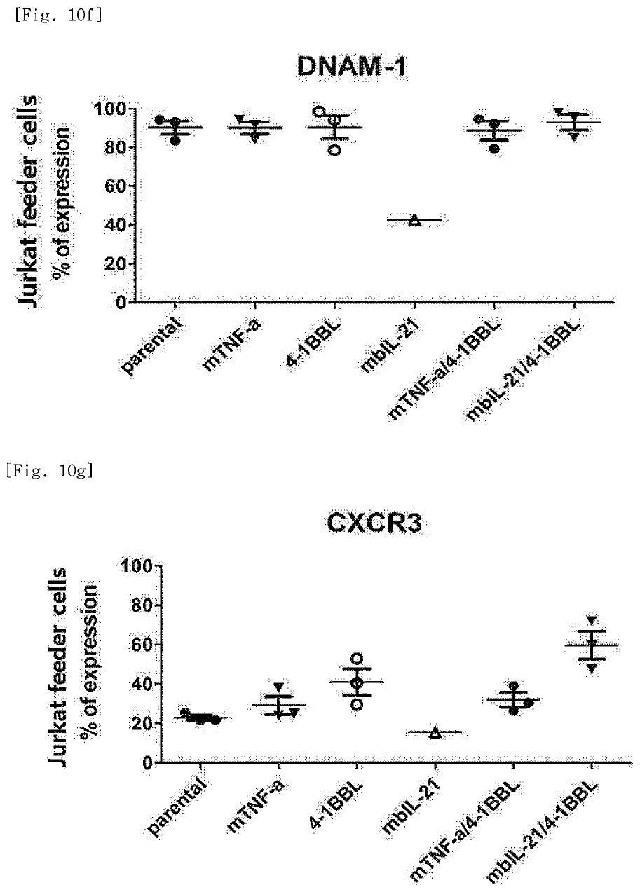

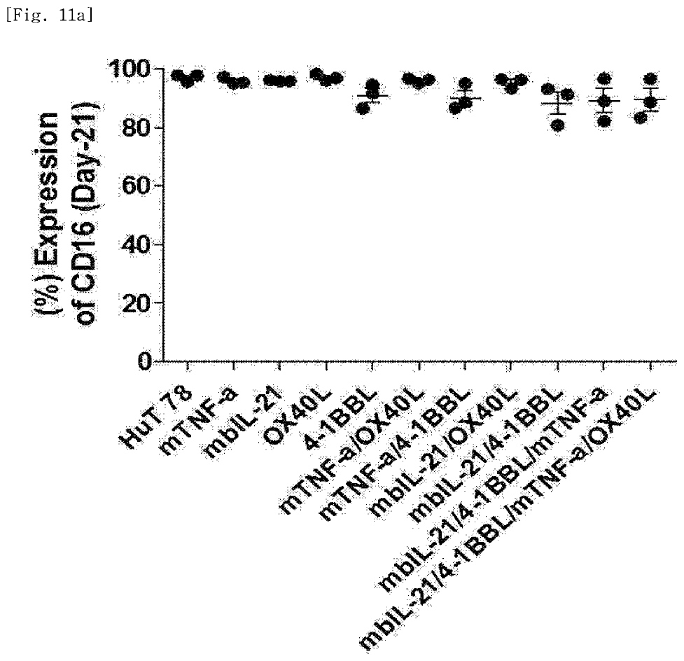

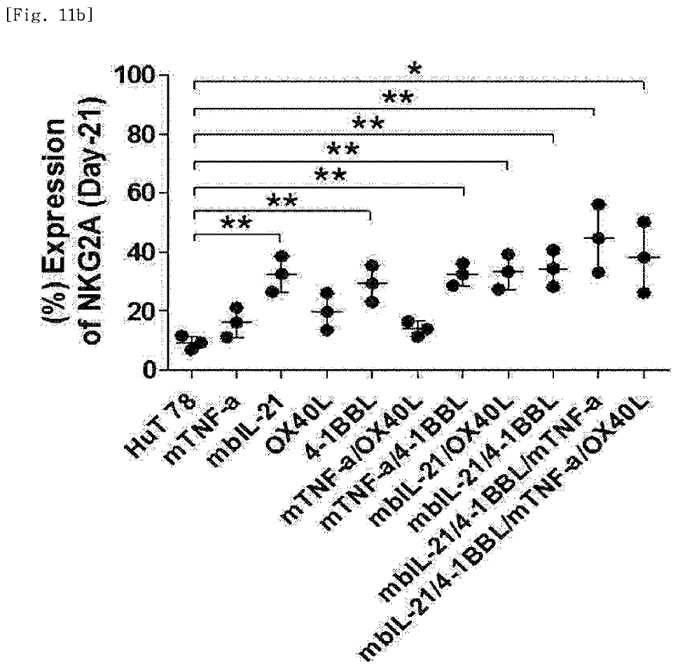

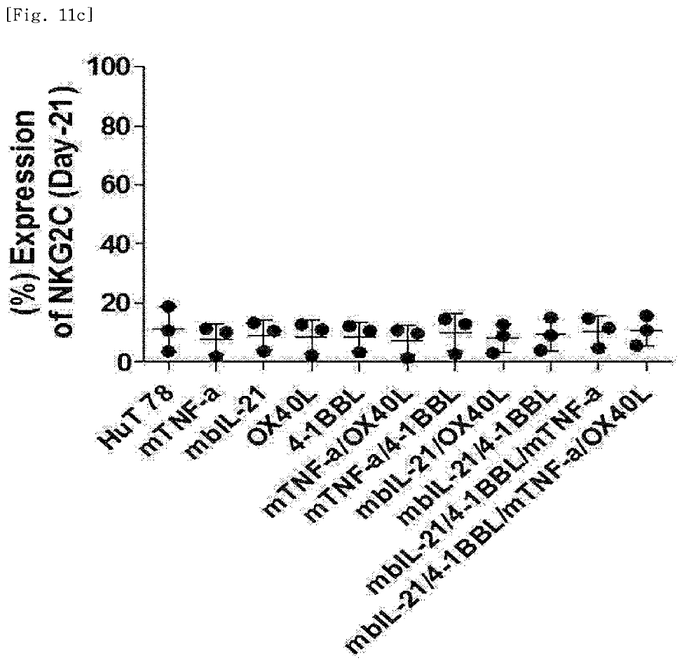

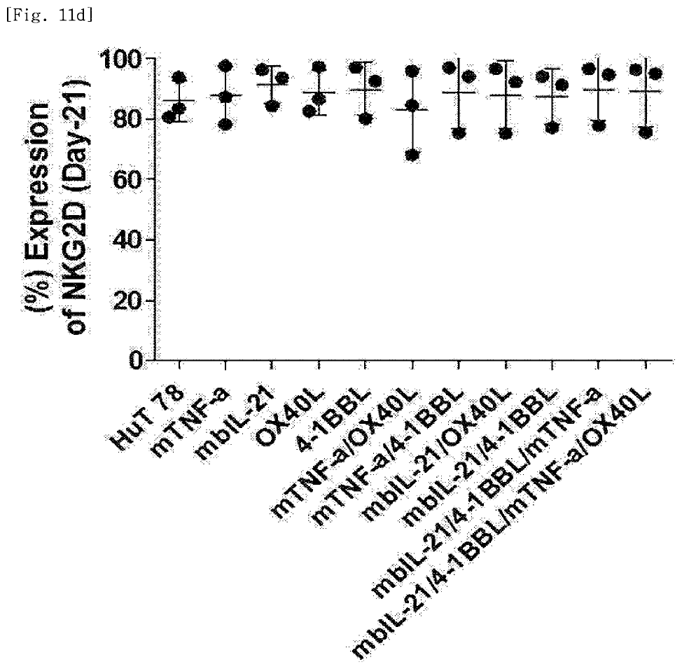

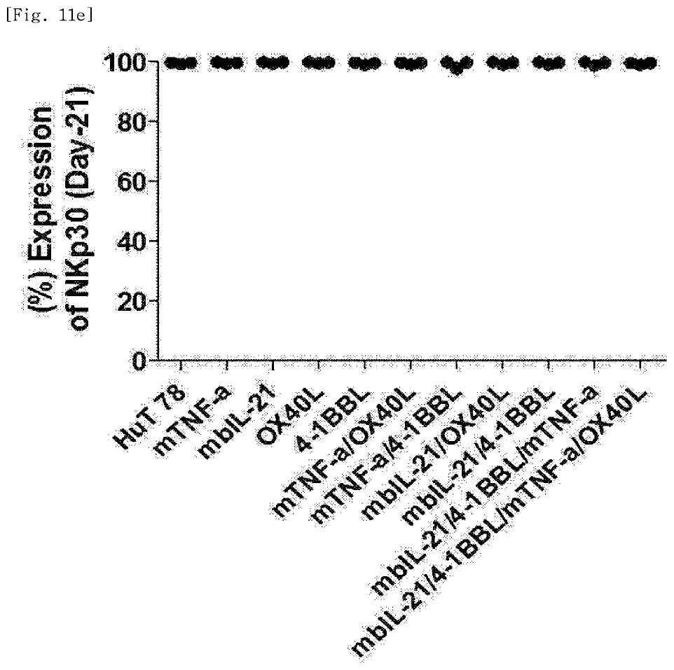

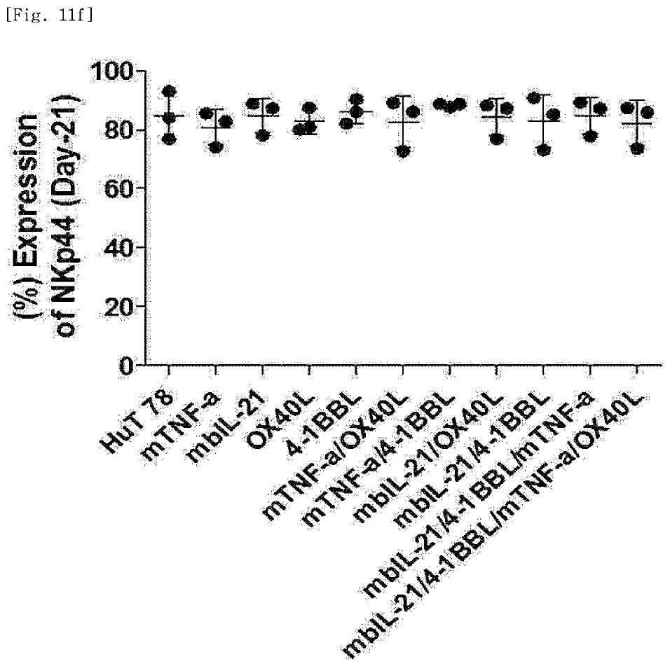

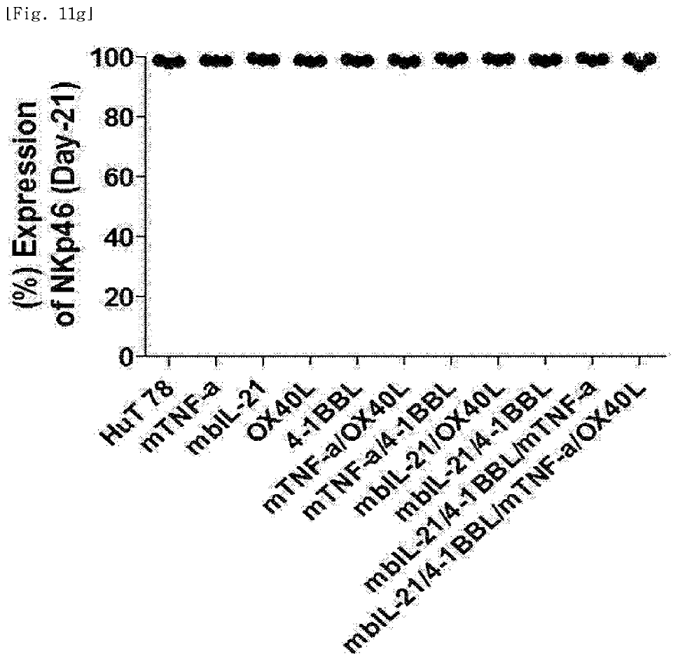

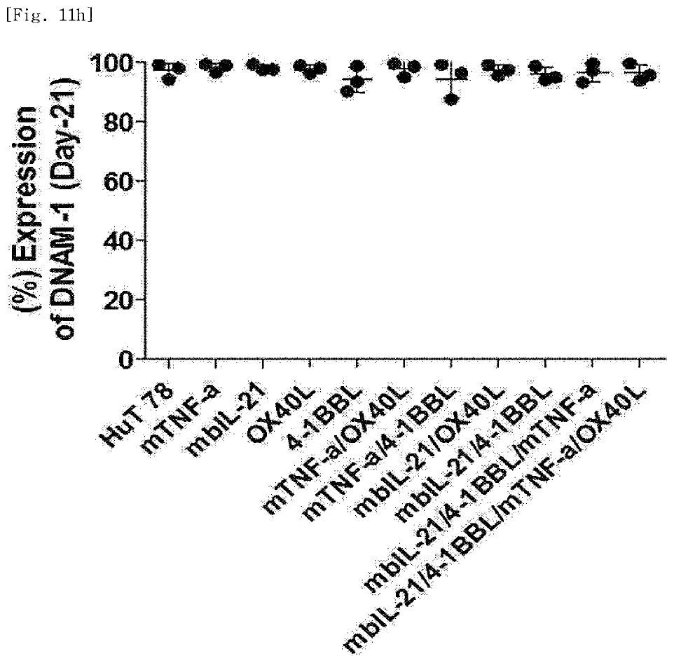

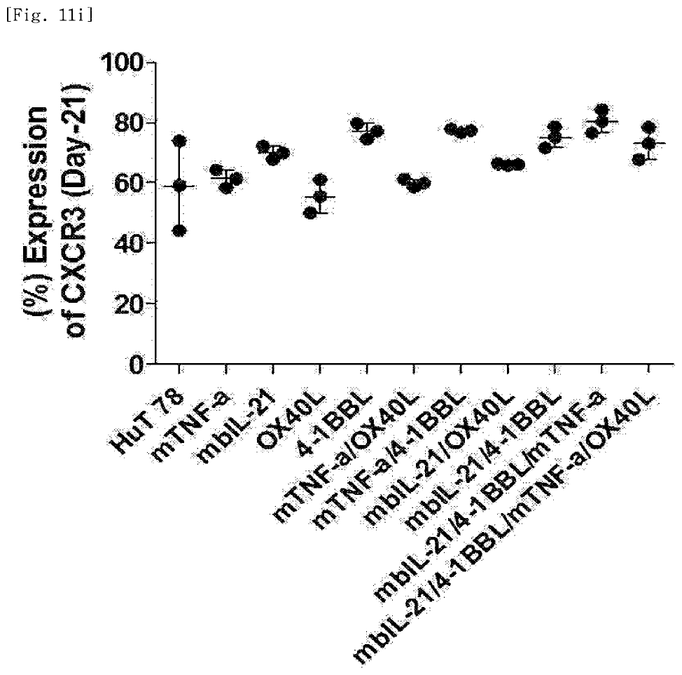

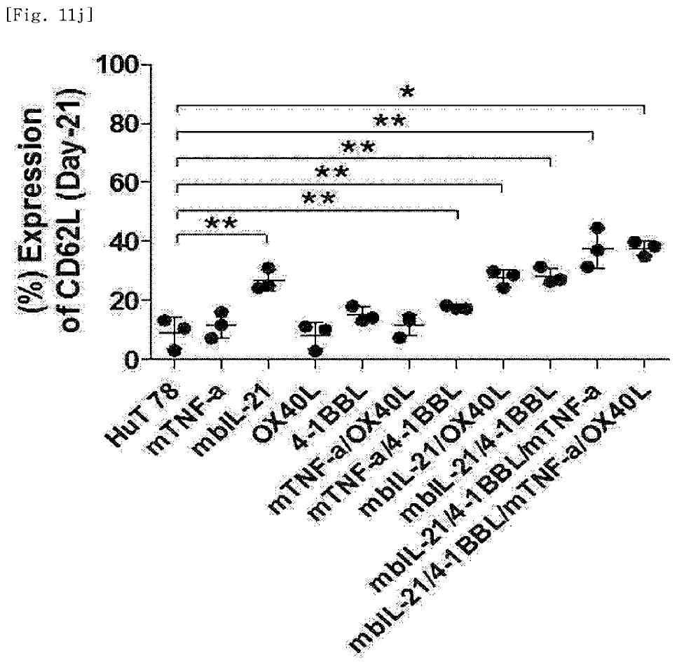

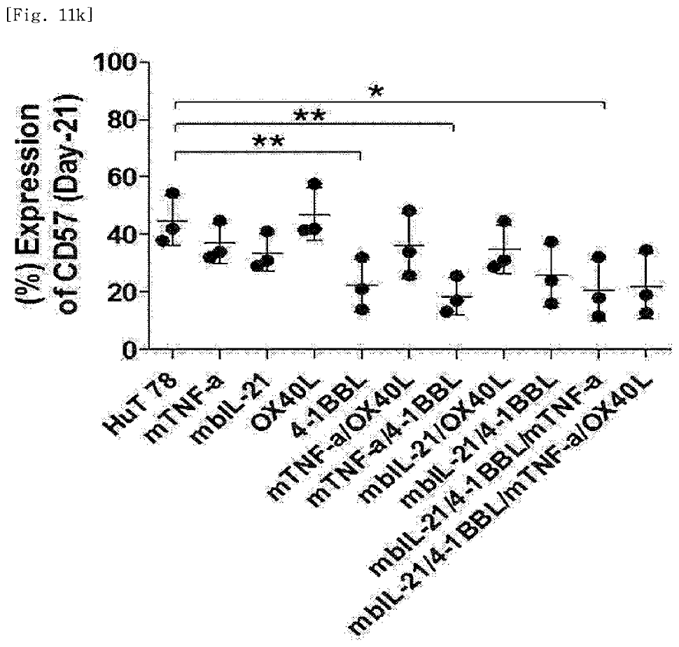

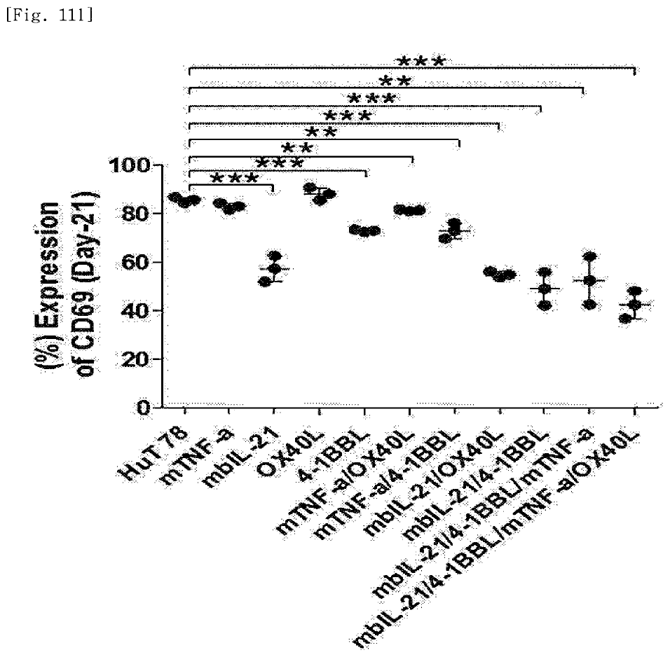

In addition, expression of representative receptors in natural killer cells was analyzed. It was identified that all natural killer cells expressed CD16 at a high level, and expressed the activation markers NKG2D, NKp30, NKp44, NKp46, and DNAM-1 at high levels, without variation among donors, under a condition of feeder cells having undergone double gene transduction, as compared with a condition where no gene has been transduced or a condition of feeder cells having undergone single gene transduction. In particular, it was possible to identify that the amount of CXCR3 was greatly increased under a condition of feeder cells having undergone double gene transduction. Therefore, it was identified that the feeder cells having undergone double gene transduction were useful feeder cells capable of increasing activation and tumor targeting of NK cells (FIGS. 8a to 11l).

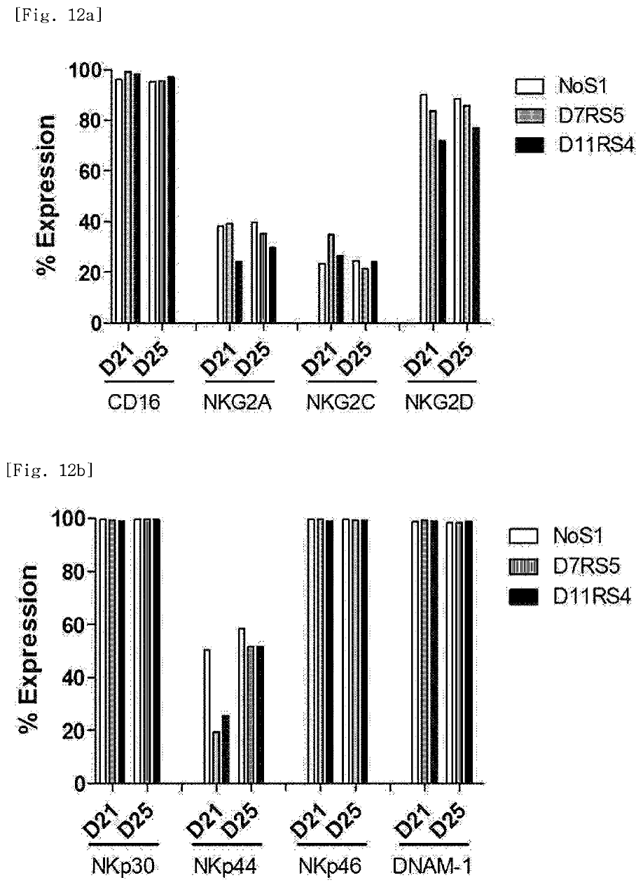

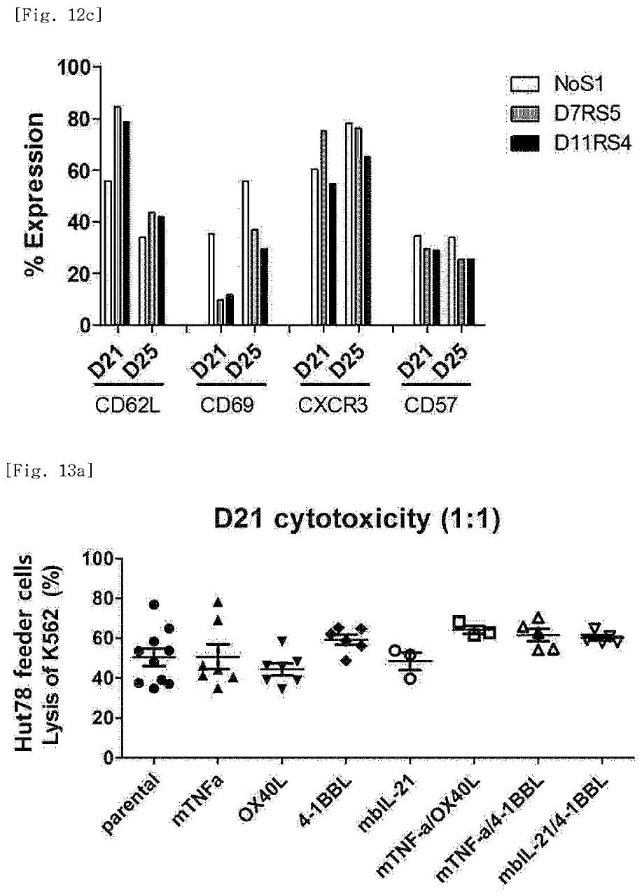

As a result of identifying receptors and activation markers of the natural killer cells cultured with repeated re-stimulation by T cell line due to gene transduction, it was identified that under both the condition where no re-stimulation was performed and the condition where repeated re-stimulation was performed, the activation markers (CD16, NKG2D, NKp30, NKp44, NKp46, DNAM-1) were expressed with no large difference even when a re-stimulation interval or a culture period increased (FIGS. 12a to 12c).

### Experimental Example 3. Identification of Abnormal Cell-Killing Activity of Natural Killer Cells Depending on Transduced Gene(s) for T Cells

1×10⁶ cells of K562 cancer cell line were placed in a 15-ml tube and centrifugation was performed. The cell pellets were suspended in RPMI1640 medium supplemented with 1 ml of 10% (v/v) FBS. Thereafter, 30 μl of 1 mM Calcein-AM (Molecular probe, C34852) was added thereto. Then, silver foil was used to block the light, and staining was performed for 1 hour in an incubator at a temperature condition of 37° C.

The Calcein-AM-stained tumor cell line was washed by adding thereto 10 ml to 15 ml of RPMI1640 medium supplemented with 10% (v/v) FBS, and centrifugation was performed. Then, the pellets were suspended in 10 ml of RPMI1640 medium supplemented with 10% (v/v) FBS so that the cells had a concentration of 1×10⁵ cells/ml. Natural killer cells were placed in a 15-ml tube at 1×10⁶ cells, and centrifugation was performed. The pellets were suspended in RPMI1640 medium supplemented with 10% (v/v) FBS at a desired ratio (1:1) with respect to K562 cancer cell line. The prepared K562 cancer cell line and natural killer cell line were mixed using 100 μl each and dispensed into a 96-well U-bottom plate (Nunc, 163320). Each well was prepared in triplicate and a mean value thereof was obtained.

To spontaneous release wells were added 100 μl of the stained K562 cancer cell line and 100 μl of RPMI1640 medium supplemented with 10% (v/v) FBS. To maximum release wells were added 100 μl of the stained K562 cancer cell line and 100 μl of triple distilled water supplemented with 2% (v/v) Triton-X 100.

In order to correct auto-fluorescence present in RPMI1640 medium supplemented with 10% (v/v) FBS and RPMI1640 medium supplemented with 2% (v/v) Triton-X 100, 200 μl of RPMI1640 medium supplemented with 10% (v/v) FBS was added to prepare a medium value; and 100 μl of RPMI1640 medium supplemented with 2% (v/v) Triton-X 100 was added to 100 μl of RPMI1640 medium supplemented with 10% (v/v) FBS, to prepare a mixed solution value of the two solutions. The auto-fluorescence was corrected by adding, to the maximum release value, the difference (A) obtained by subtracting the mixed solution value from the medium value.

The light was blocked and reaction was allowed to occur for 4 hours in an incubator at a temperature condition of 37° C., and then the plate was centrifuged at 2,000 rpm for 3 minutes. The supernatant was dispensed at 100 μl each into 96-well black plate (Nunc, 237108). Fluorescence values (OD_{480/535} nm) were measured using a fluorescence plate reader (Perkin Elmer, VICTOR X3), and tumor cell-killing ability of natural killer cells was calculated using the following Expression II.

### [Expression II]

% of killing=(Sample well mean fluorescence value−Spon well mean fluorescence value)/{(Max well mean fluorescence value+A)−Spon well mean fluorescence value}×100

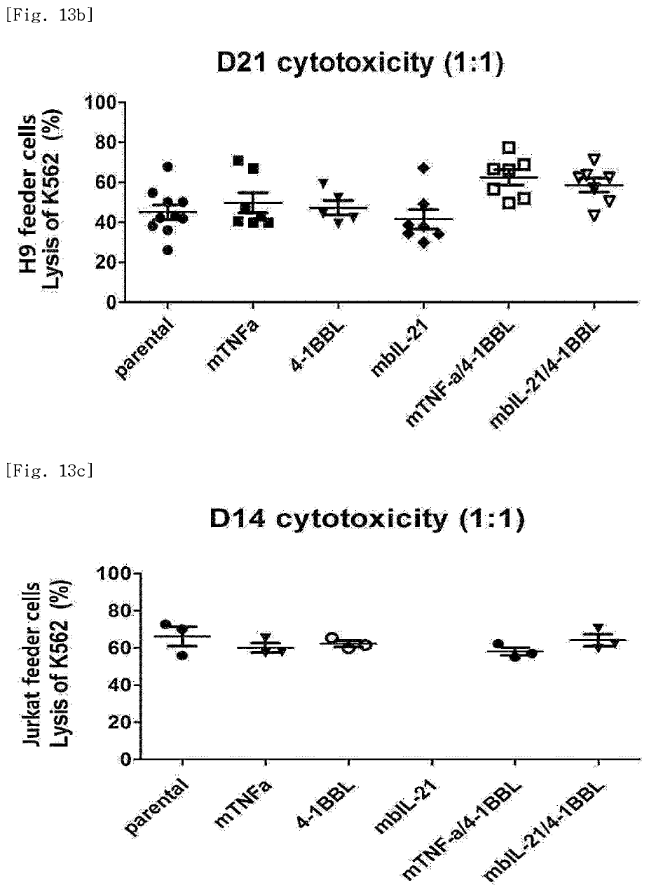

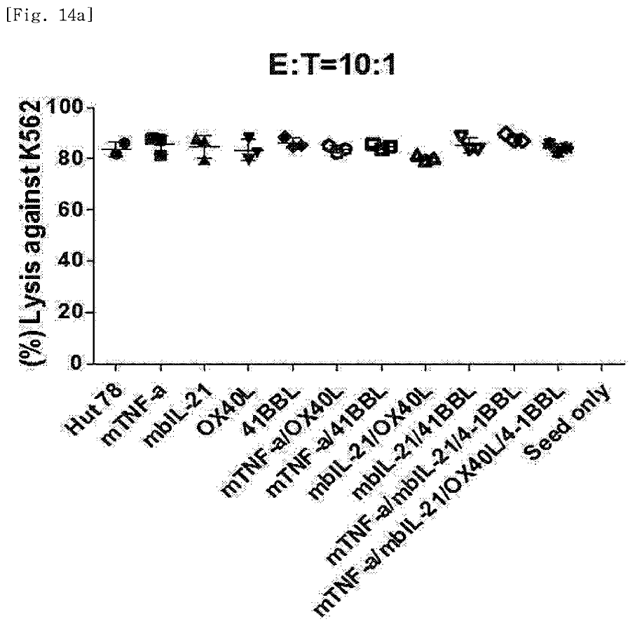

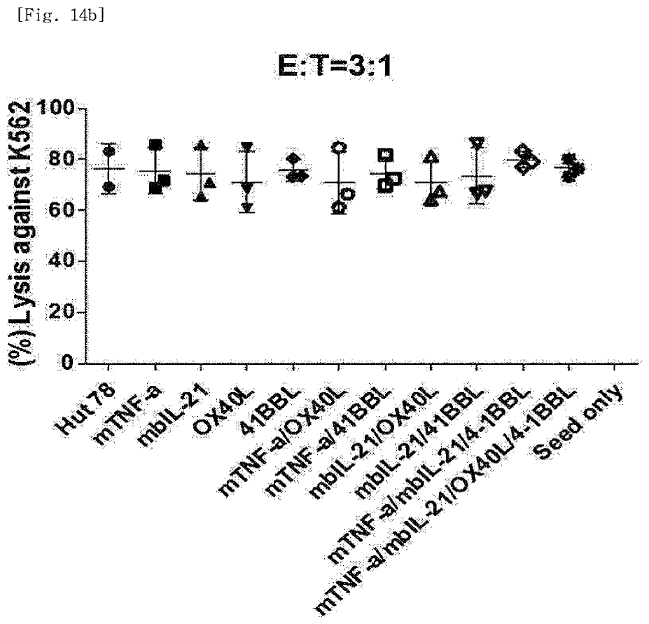

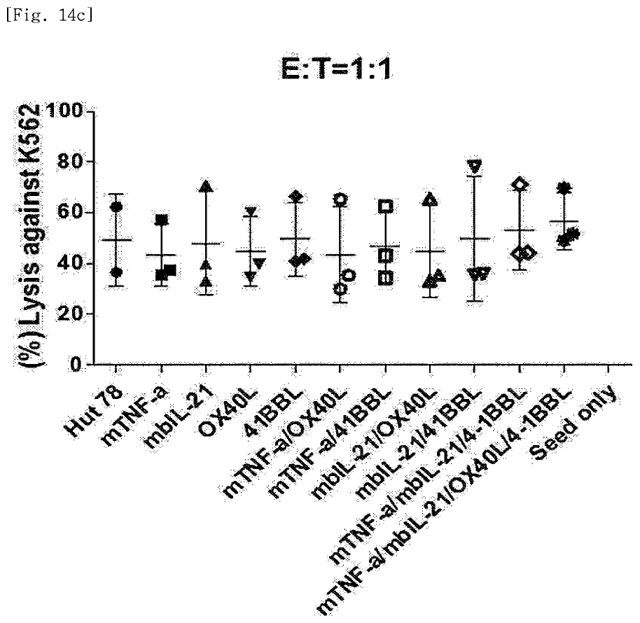

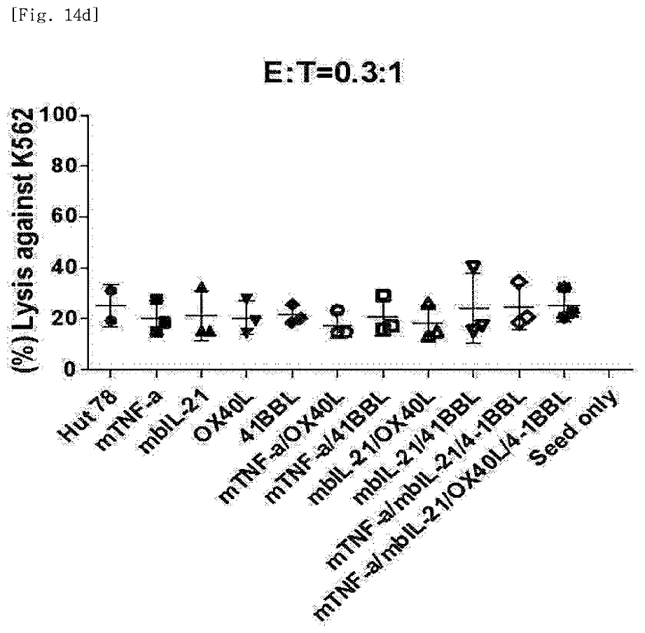

The natural killer cells cultured with various feeder cells were reacted with K562 cancer cell line to measure their direct cell-killing activity. As compared with other conditions, the killing activity against the K562 cancer cell line was increased on average under a condition of feeder cells having undergone double gene transduction (FIGS. 13a to 14d).

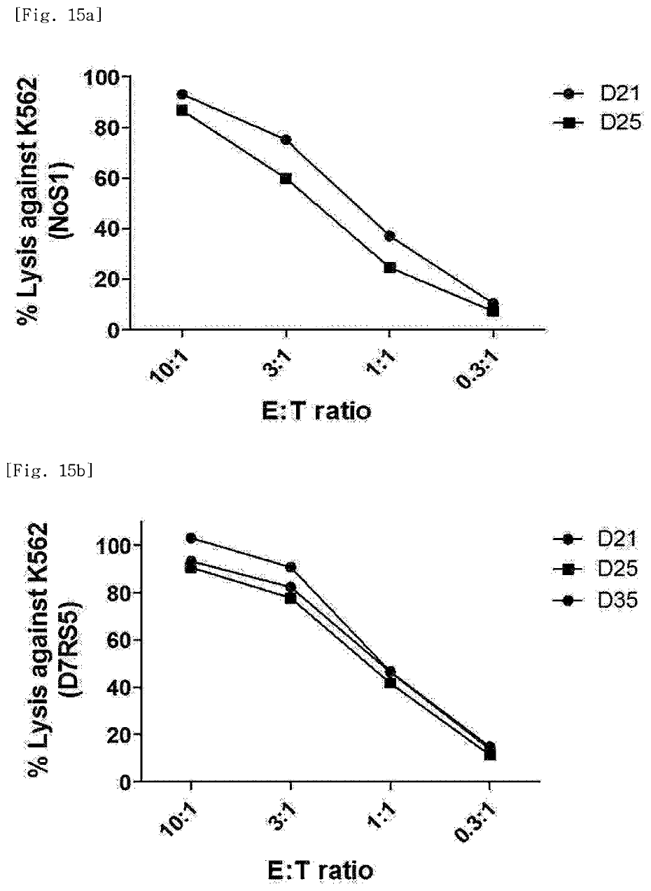

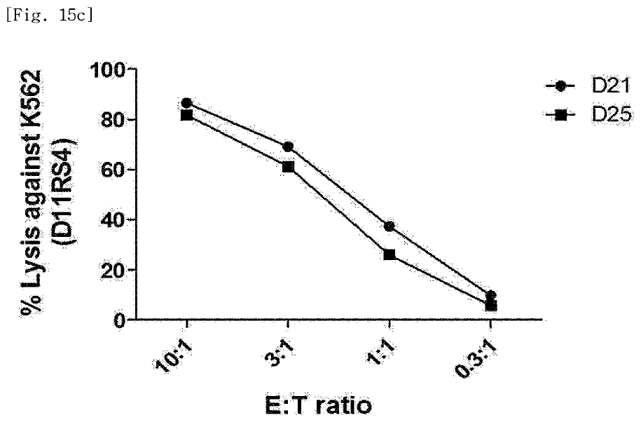

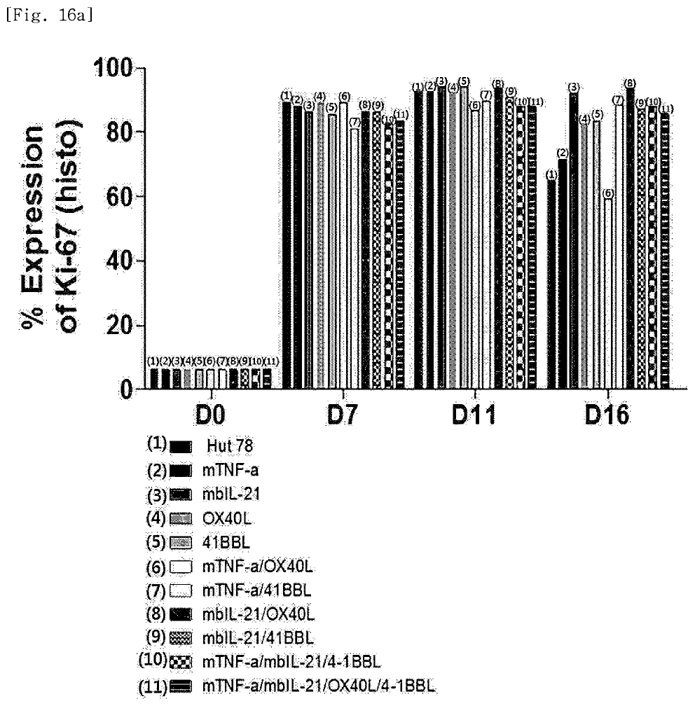

The natural killer cells cultured with re-stimulation by T cell line due to gene transduction were reacted with K562 cancer cell line to measure their direct cell-killing activity. It was identified that under both the condition where no re-stimulation was performed and the condition where repeated re-stimulation was performed, the killing activity against K562 was well maintained even when a culture period was prolonged and fold expansion was increased (FIGS. 15a to 15c).

### Experimental Example 4. Analysis of Proliferation Marker (Ki-67) in Natural Killer Cells Depending on Transduced Gene(s) for T Cells

The natural killer cells cultured for 21 days or the natural killer cells cultured with repeated re-stimulation were collected and centrifuged at 1,200 rpm for 5 minutes. The culture was removed by suction. Dilution with 1 ml of FACS buffer was performed and the cell number was measured. Dilution with FACS buffer was performed so as to give a concentration of 5×10⁶ cells/ml. 100 μl of the diluted cell solution was added to each 5-ml FACS tube (Falcon, 352052), and proliferation marker analysis was performed with the following antibodies.
Tube 1: Anti-human CD3-FITC (BD) Pharmingen, 555332), anti-human CD56-PE-Cy7 (BD Pharmingen, 557747), 7-AAD (Beckman Coulter, Inc., A07704)
Tube 2: Anti-human CD3-FITC (BD Pharmingen, 555332), anti-human CD56-PE-Cy7 (BD Pharmingen, 557747), 7-AAD (Beckman Coulter, Inc., A07704)
Tube 3: FITC Mouse IgG1 k isotype control (BD Pharmingen, 555748), PE-Cy7 Mouse IgG1 k isotype control (BD Pharmingen, 557872)

The cells in the above tubes were stained at refrigeration temperature for 30 minutes. Thereafter, 2 ml of FACS buffer was added to the stained cells, and centrifugation was performed at 1,500 rpm for 5 minutes. The supernatant was removed, and 2 ml of FACS buffer was added again. Centrifugation was performed at 1,500 rpm for 5 minutes. The supernatant was removed again, and placed and suspended in 200 μl of fixation/permeabilization buffer (eBioscience, 00-5521-00). Then, reaction was allowed to occur at refrigeration temperature for 30 minutes or longer. 2 ml of 1×Perm/wash buffer was added and centrifugation was performed at 1,500 rpm for 5 minutes. The supernatant was removed, and 2 ml of FACS buffer was added again. Centrifugation was performed at 1,500 rpm for 5 minutes. Anti-human Ki-67-V450 (BD Pharmingen, 561281) was added to the tube 1, and V450 mouse IgG1 k isotype control (BD Pharmingen, 561504) was added to the tubes 2 and 3. Staining was performed at room temperature for 30 minutes.

Thereafter, 2 ml of 1×Perm/wash buffer was added to the stained cells, and centrifugation was performed at 1,500 rpm for 5 minutes. The supernatant was removed, and 2 ml 1×Perm/wash buffer was added again. Centrifugation was performed at 1,500 rpm for 5 minutes. The supernatant was removed again, and placed and suspended in 200 μl of fixation buffer (BD, 554655). Then, FACS LSRII Fortessa (BD) was used to examine the proliferation marker in the cells.

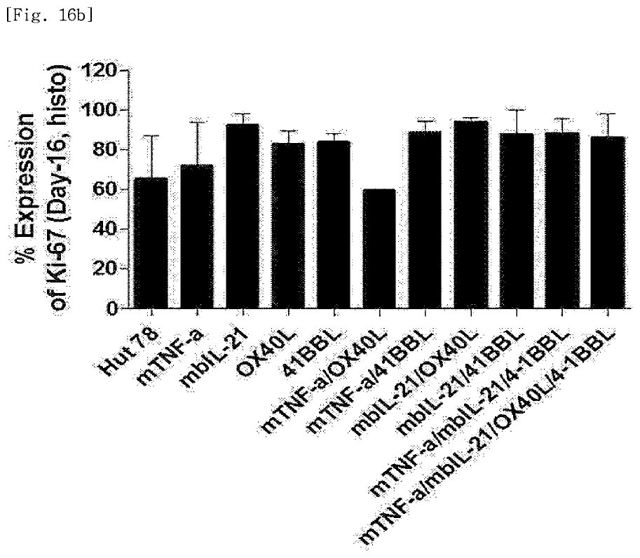

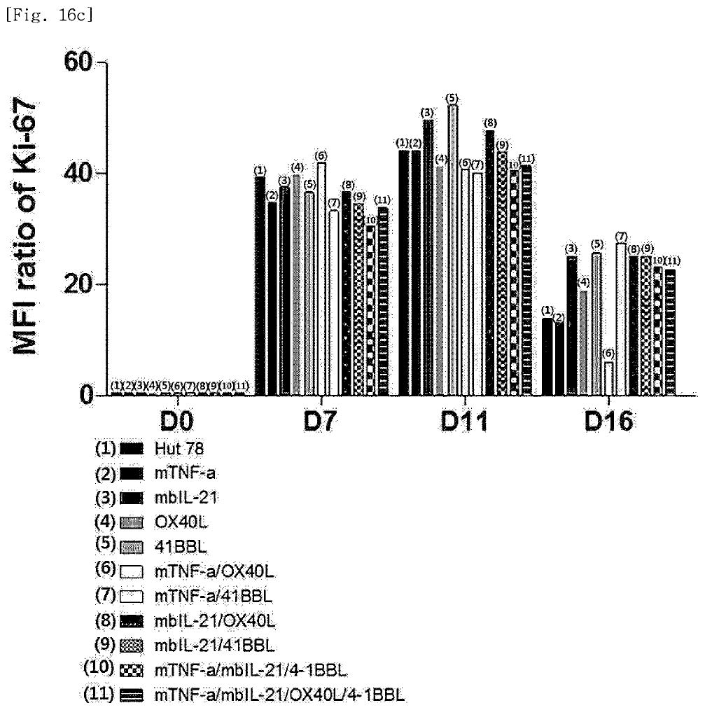

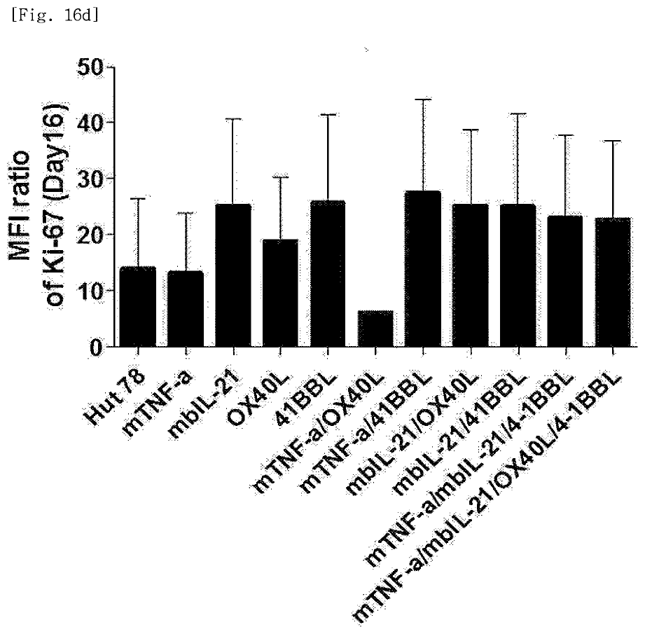

It was identified that expression of Ki-67 in the natural killer cells cultured with HuT 78 cell lines into which one or more genes have been transduced was maintained at a relatively high level, except for Hut78-mTNF-α/OX40L, as compared with the HuT 78 cell line having no gene transduction (FIGS. 16a to 16d).

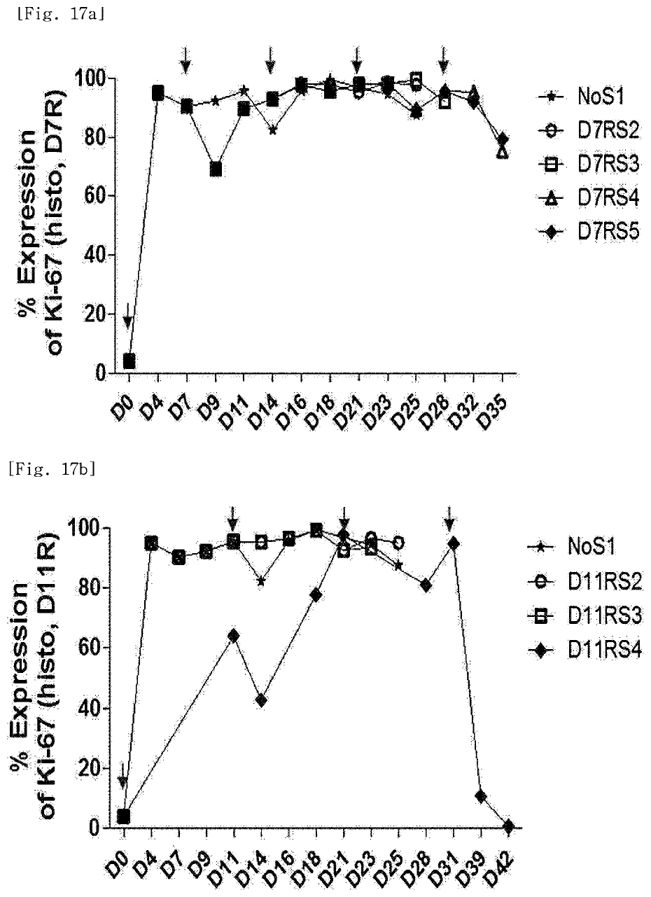

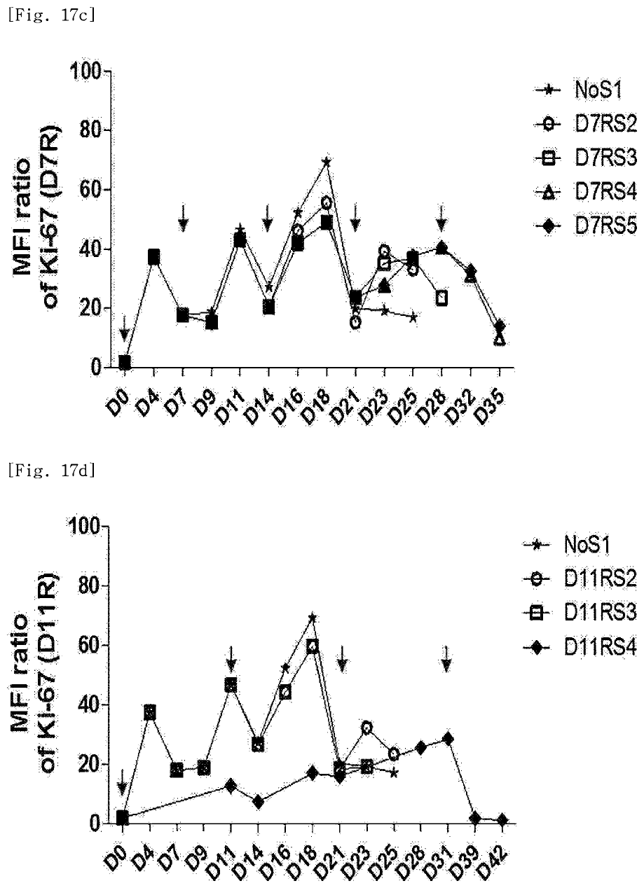

Analysis was performed on the proliferation marker in the natural killer cells cultured through re-stimulation by Hut78-mTNF-α/4-1BBL/mbIL-21 feeder cells. As a result, it was identified that expression of Ki-67 which had been decreasing increased with stimulation by feeder cells at 7-day or 11-day intervals. In addition, it was identified that expression of Ki-67 could be sustained through repeated re-stimulation (FIGS. 17a to 17d).

### Experimental Example 5. Analysis of Co-Stimulatory Molecules in Natural Killer Cells Depending on Transduced Gene(s) for T Cells

The natural killer cells cultured for 21 days or the natural killer cells cultured with repeated re-stimulation were collected and centrifuged at 1,200 rpm for 5 minutes. The culture was removed by suction. Dilution with 1 ml of FACS buffer was performed and the cell number was measured. Dilution with FACS buffer was performed so as to give a concentration of 5×10⁶ cells/ml. 100 μl of the diluted cell solution was added to each 5-ml FACS tube (Falcon, 352052), and phenotypic analysis was performed with the following antibodies.
Tube 1: Anti-human CD3-FITC (BD Pharmingen, 555332), anti-human CD25-PE (BD Pharmingen, 555432), anti-human CD56-PE-Cy5 (BD Pharmingen, 555517) or anti-human CD56-PE Cy7 (BD Pharmingen, 557747), anti-human CD56-BV421 (BD Pharmingen, 562751)
Tube 2: Anti-human CD3-FITC, anti-human TNFRII-PE (R&D Systems, FAB226P), anti-human CD56-PE-Cy5 or anti-human CD56-PE-Cy7, anti-human CD56-BV421
Tube 3: Anti-human CD3-FITC, anti-human 4-1BB-PE (BD Pharmingen, 559446), anti-human CD56-PE-Cy5 or anti-human CD56-PE-Cy7, anti-human CD56-BV421
Tube 4: Anti-human CD3-FITC, PE mouse IgG1 k isotype control (BD Pharmingen, 555749), anti-human CD56-PE-Cy5 or anti-human CD56-PE-Cy7, anti-human CD56-BV421
Tube 5: FITC mouse IgG1 k isotype control (BD) Pharmingen, 555748), PE mouse IgG1 k isotype control, PE-Cy5 mouse IgG1 k isotype control (BD Pharmingen, 555750) or PE-Cy7 mouse IgG1 k isotype control (BD Pharmingen, 557872), BV421 mouse IgG1 k isotype control (BD Pharmingen, 562438)

In Tube 1, Staining with anti-human CD56 was performed by selecting one of three fluorescences, and thus the same fluorescence was selected for CD56 of Tubes 2 to 4, and isotype control of Tube 5.

The cells in the above tubes were stained at refrigeration temperature for 30 minutes. Thereafter, 2 ml of FACS buffer was added to the stained cells, and centrifugation was performed at 1,500 rpm for 5 minutes. The supernatant was removed, and 2 ml of FACS buffer was added again. Centrifugation was performed at 1,500 rpm for 5 minutes. The supernatant was removed again, and placed and suspended in 200 μl of fixation buffer (BD, 554655). Then, FACS LSRII Fortessa (BD)) was used to perform identification of the cells and examination for co-stimulatory molecules.

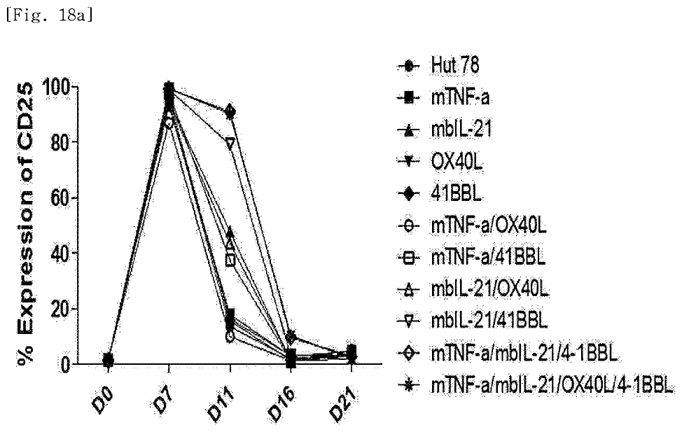

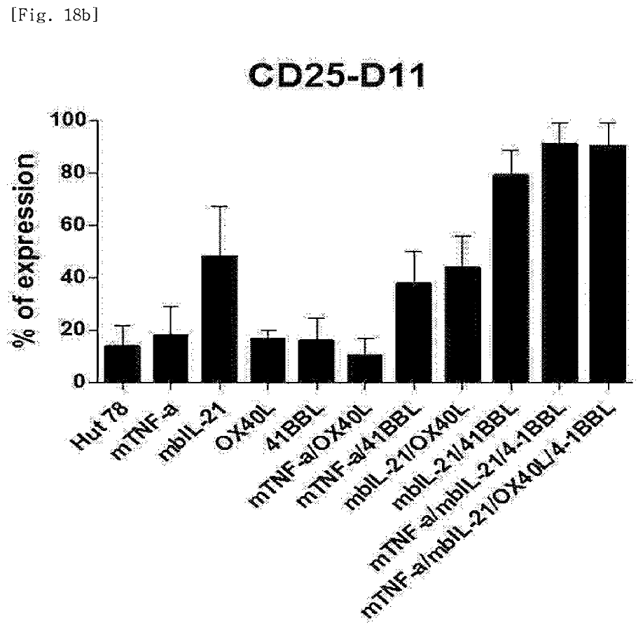

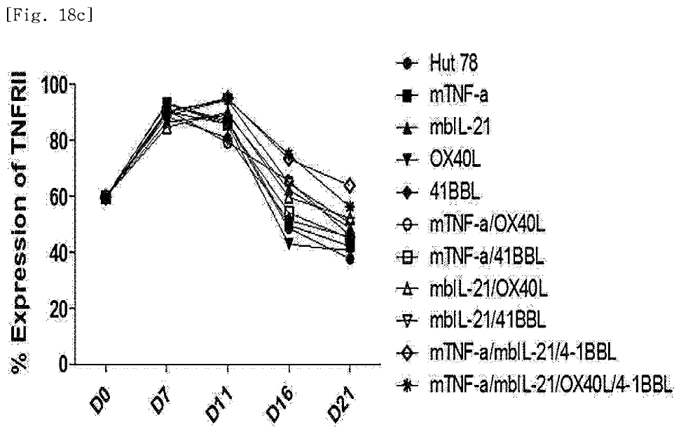

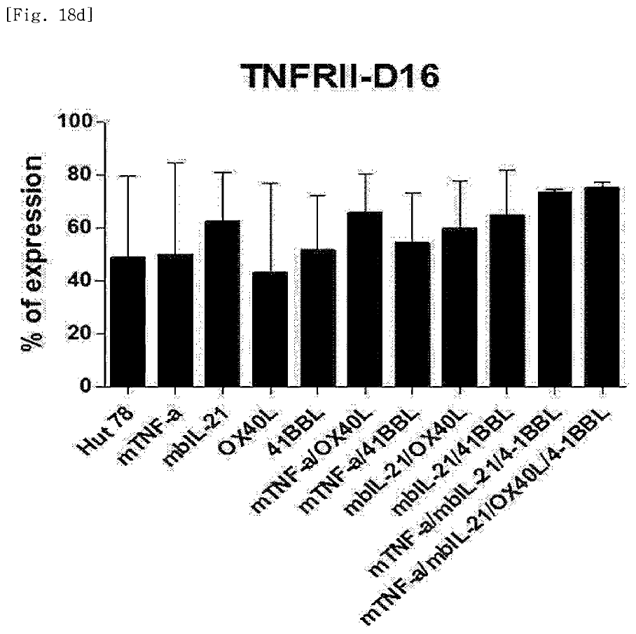

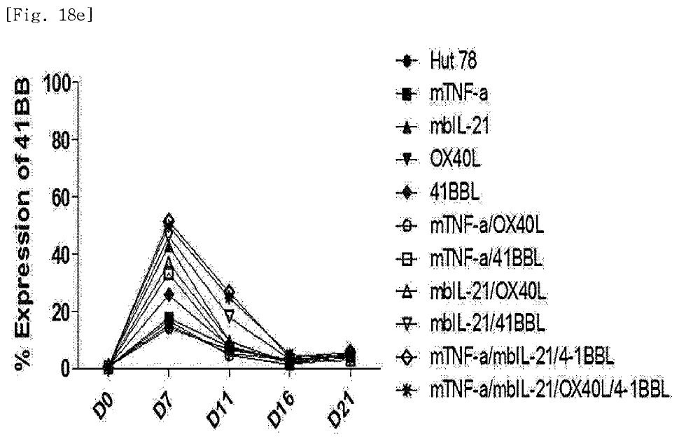

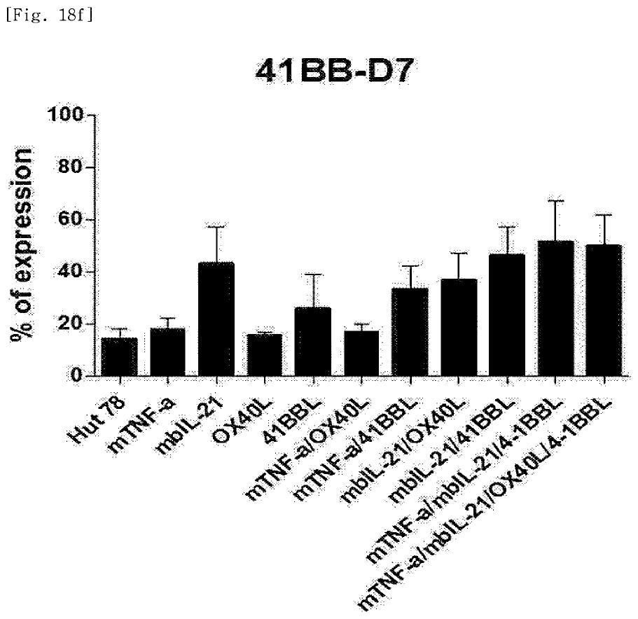

Analysis was performed on co-stimulatory molecules in the natural killer cells cultured for 21 days using Hut78 cell lines having various gene combinations. As a result, it was identified that expression of the co-stimulatory molecules in the natural killer cells cultured with the Hut78 cell lines into which one or more genes have been transduced is maintained at a relatively high level as compared with the non-transgenic Hut78 cell line. In particular, it was identified that the cell lines into which a single gene or combinations of multiple genes including the mbIL-21 gene have been transduced exhibited higher expression of co-stimulatory molecules than other cell lines (FIGS. 18a to 18f).

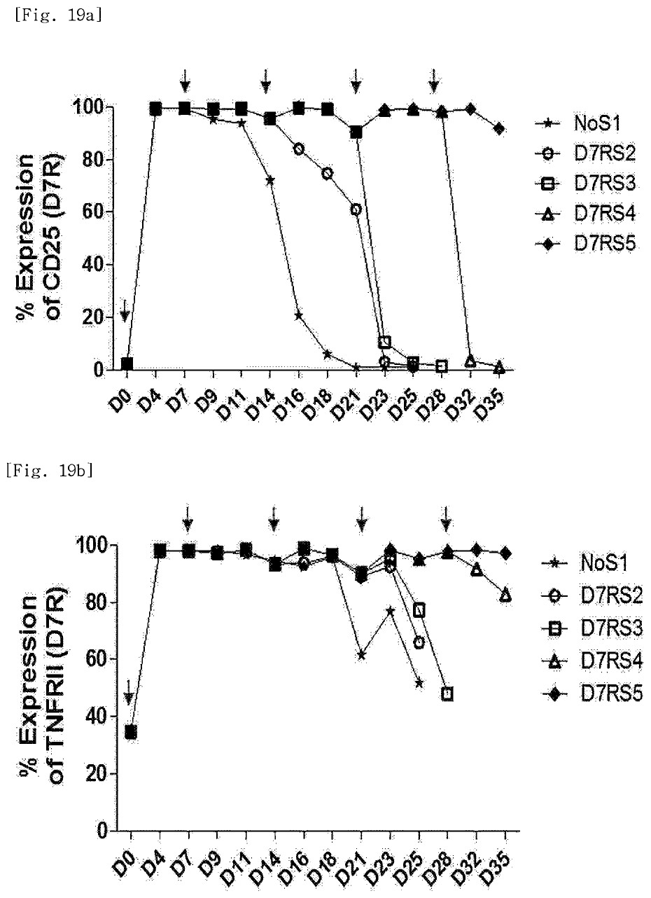

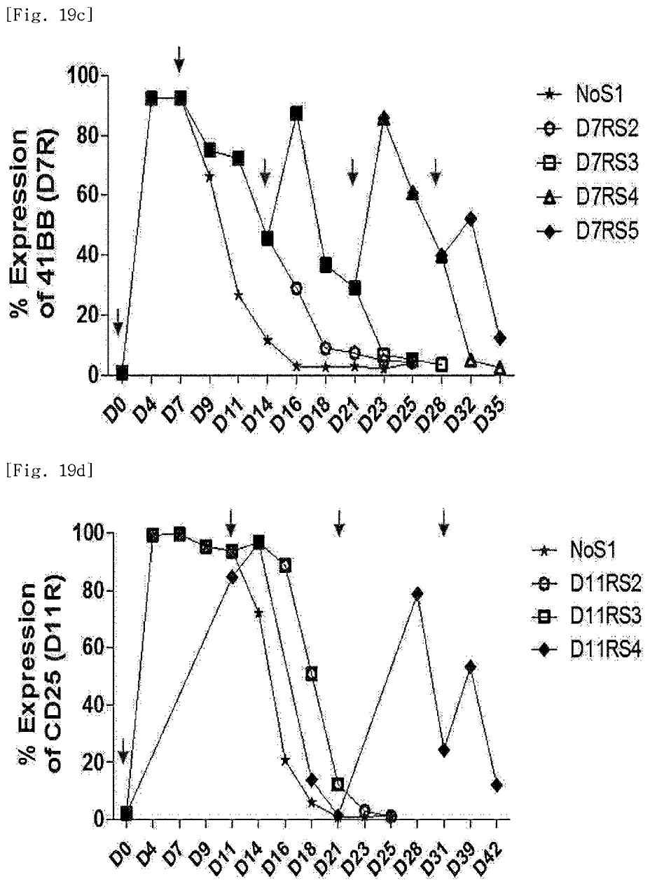

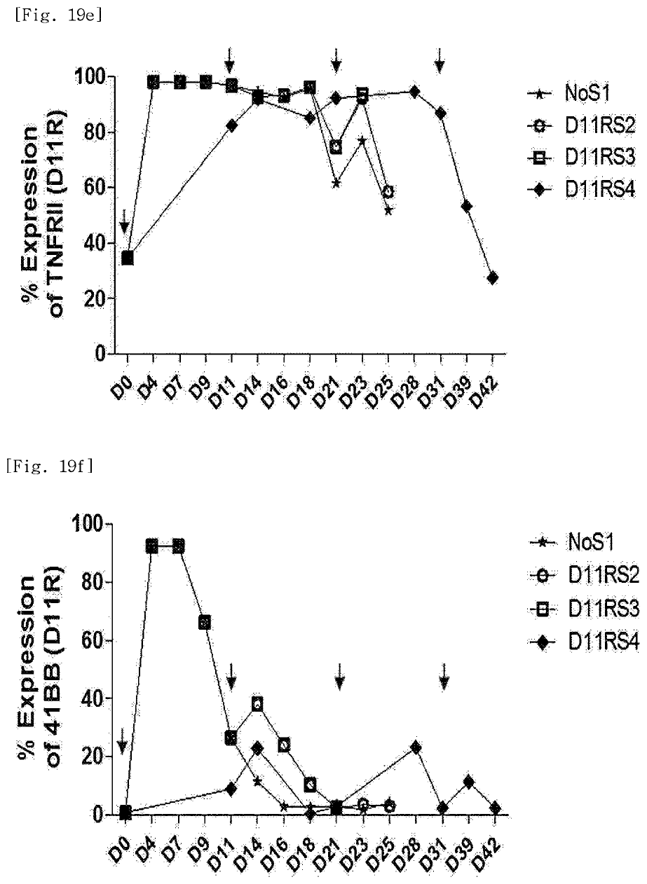

Analysis was performed on co-stimulatory molecules in the natural killer cells cultured after one stimulation by Hut78-mTNF-α/4-1BBL/mbIL-21 cell line on day 0 of culture or the natural killer cells cultured through repeated re-stimulation by Hut78-mTNF-α/4-1BBL/mbIL-21 cell line at 7-day or 11-day intervals. As a result, expression of the co-stimulatory molecules increased and then remained in a decreased state, unless additional re-stimulation is performed after the first stimulation. On the other hand, expression of the co-stimulatory molecules was continued during the culture period while repeatedly showing that upon repeated stimulation by feeder cells, expression of the co-stimulatory molecules increased at a time point of stimulation and then decreased after a certain time elapsed (FIGS. 19a to 19f).

### Experimental Example 6. Identification of Cytokine-Secretion of Natural Killer Cells Depending on Transduced Gene(s) for T Cells

2.5×10⁶ cells of K562 cancer cell line were placed in a 15-ml tube and centrifuged. The cell pellets were suspended in 1 ml of RPMI1640 medium supplemented with 10% (v/v) FBS.

Natural killer cells were placed at 2.5×10⁶ cells in a 15-ml tube and centrifuged. The cell pellets were suspended in RPMI1640 medium supplemented with 1 ml of 10% (v/v) FBS. In order to identify, within the cells, cytokines to be secreted out of the cells, the protein transport inhibitors Golgiplug (BD, 555029) and Golgistop (BD, 554724) were added thereto.

The prepared cells were dispensed into a 96-well U-bottom plate (Nunc, 163320), in which the natural killer cells and the medium were added in 100 μl each to well 1, and the K562 cancer cell line and the natural killer cell line were mixed using 100 μl each and added to wells 2 and 3.
Wells 1, 2: Anti-human CD107a-APC (BD Pharmingen, 560664)
Well 3: APC mouse IgG1 k isotype control (BD Pharmingen, 555751)

The light was blocked and reaction was allowed to occur for 4 hours in an incubator at a temperature condition of 37° C., and then the plate was centrifuged at 2,000 rpm for 3 minutes. The supernatant was removed and cytokine-secreting ability analysis was performed with the following antibodies.

Wells 1, 2, 3: Anti-human CD3-PerCP-Cy5.5 (eBioscience, 45-0036-42), anti-human CD56-APC-Cy7 (eBioscience, 47-0567-42), 7-AAD (Beckman Coulter, Inc., A07704)

The plate was stained at refrigeration temperature for 30 minutes. Thereafter, 100 μl of FACS buffer was added to the stained cells, and centrifugation was performed at 2,000 rpm for 3 minutes. The supernatant was removed and 200 μl FACS buffer was added again thereto. Centrifugation was performed at 2,000 rpm for 3 minutes. The supernatant was removed again, and placed and suspended in 200 μl of fixation/permeabilization buffer (BD Pharmingen, 554714). Then, reaction was allowed to occur at refrigeration temperature for 30 minutes or longer.

200 μl of 1×Perm/wash buffer was added thereto, and centrifugation was performed at 1,500 rpm for 5 minutes. The supernatant was removed, and 2 ml of FACS buffer was added again. Centrifugation was performed at 2,000 rpm for 3 minutes. Anti-human IFN-γ-FITC (BD Pharmingen, 554700), anti-human TNFα-PE-Cy7 (eBioscience, 25-7349-82) were added to wells 1 and 2, and FITC mouse IgG1 k isotype control (BD) Pharmingen, 555748), PE-Cy7 mouse IgG1 k isotype control (BD Pharmingen, 557872) were added to well 3. Staining was performed at refrigeration temperature for 30 minutes.

Thereafter, 100 μl of 1×Perm/wash buffer was added to the stained cells, and centrifugation was performed at 2,000 rpm for 3 minutes. The supernatant was removed and 100 μl of 1×Perm/wash buffer was added again thereto. Centrifugation was performed at 2,000 rpm for 3 minutes. The supernatant was removed again, and placed and suspended in 200 μl of fixation buffer (BD, 554655). Then, FACS LSRII Fortessa (BD) was used to examine cytokine-secreting ability of the cells.

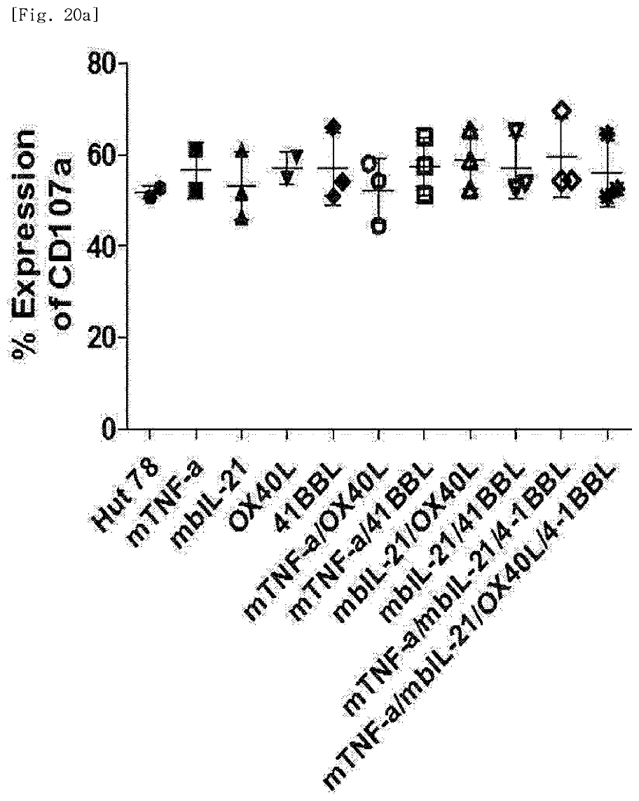

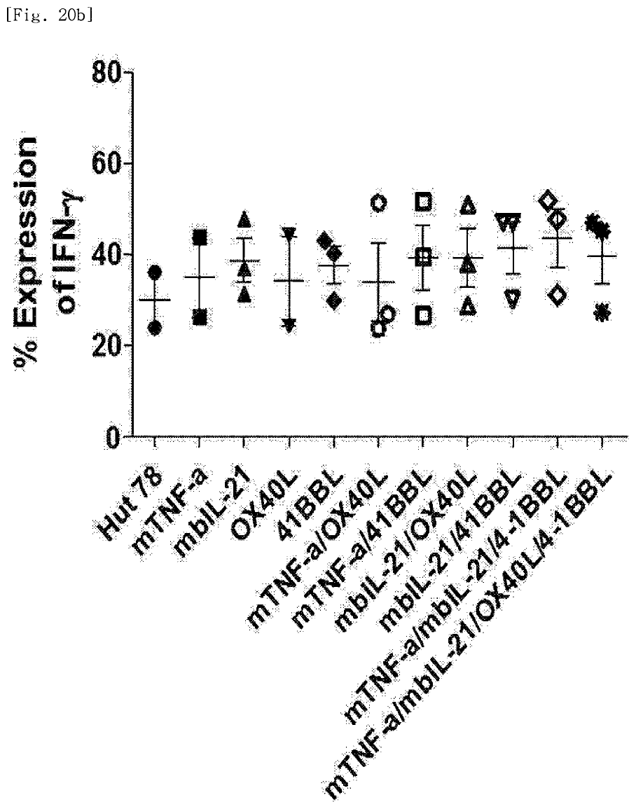

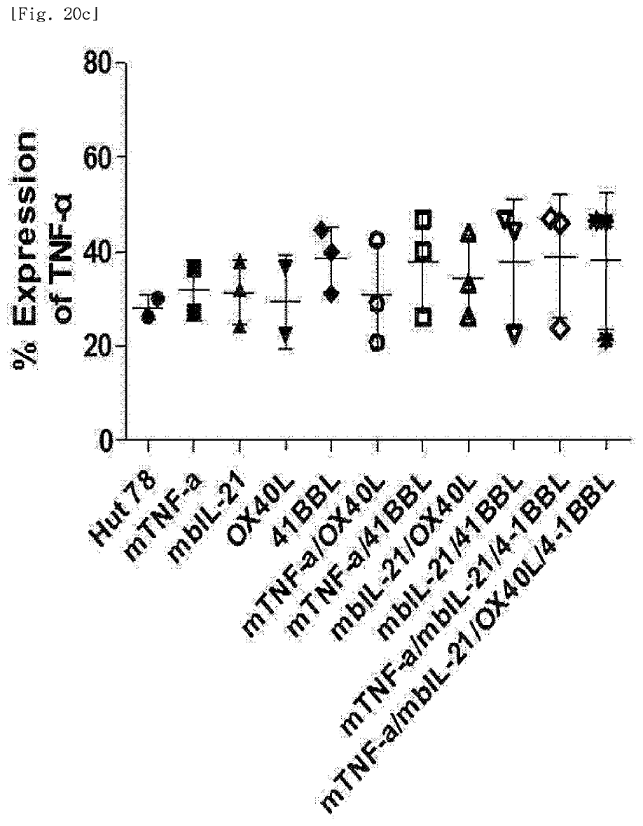

Measurements were performed on the cytokine-secreting ability, against K562 cancer cell line, of the natural killer cells cultured for 21 days using Hut78 cell lines having various gene combinations. As a result, it was identified that when being cultured with cell lines into which one or more genes have been transduced, a similar level of cytokine-secreting ability was observed despite large difference in expansion fold, as compared with the cell line having no gene transduction (FIGS. 20a to 20c).

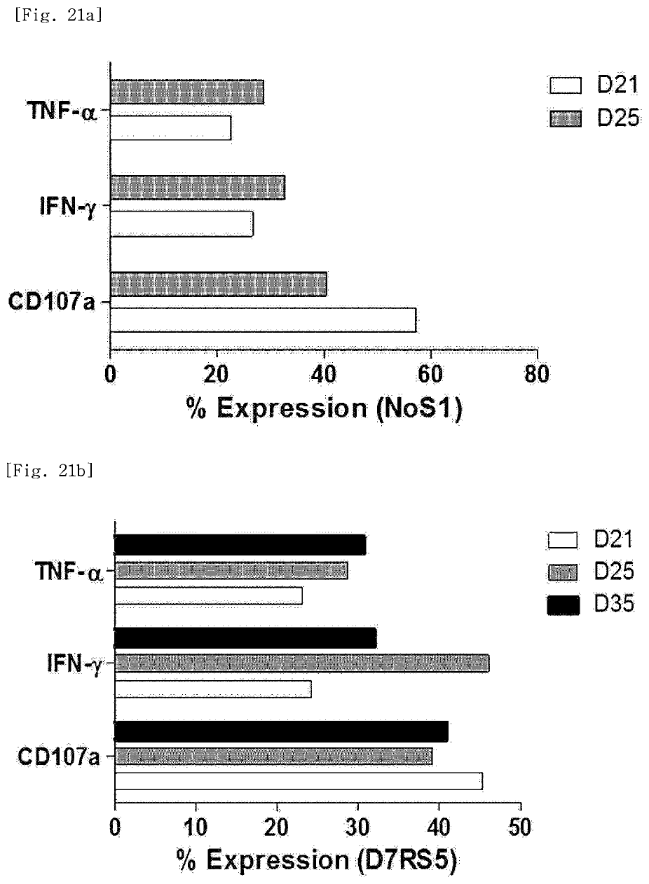

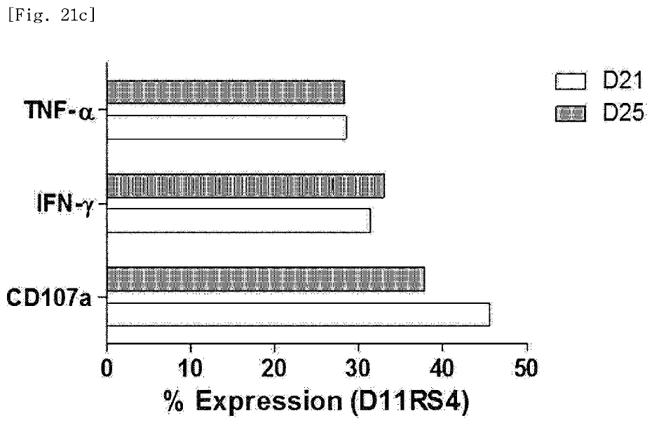

The cytokinesecretion of the natural killer cells cultured after one stimulation by Hut78-mTNF-α/4-1BBL/mbIL-21 cell line on day 0 of culture or the natural killer cells cultured through repeated re-stimulation by Hut78-mTNF-α/4-1BBL/mbIL-21 cell line at 7-day or 11-day intervals was measured by reaction with K562 cancer cell line. It was identified that as compared with the natural killer cells cultured with one stimulation, the cytokine-secreting ability against K562 was maintained, without large difference, in the natural killer cells cultured with repeated re-stimulation, despite a long culture period and high expansion fold (FIGS. 21a to 21c).

1. A genetically modified CD4+ T cell that expresses at least one gene selected from the group consisting of 4-1BBL gene, mbIL-21 gene, OX40L gene, and mTNF-α gene.

2. The genetically modified CD4+ T cell of claim 1, wherein the 4-1BBL gene has a nucleotide sequence encoding the amino acid sequence represented by SEQ ID NO: 1.

3. The genetically modified CD4+ T cell of claim 2, wherein the nucleotide sequence encoding the amino acid sequence represented by SEQ ID NO: 1 is the nucleotide sequence represented by SEQ ID NO: 2.

4. The genetically modified CD4+ T cell of claim 1, wherein the mbIL-21 gene has a nucleotide sequence encoding the amino acid sequence represented by SEQ ID NO: 3.

5. The genetically modified CD4+ T cell of claim 4, wherein the nucleotide sequence encoding the amino acid sequence represented by SEQ ID NO: 3 is the nucleotide sequence represented by SEQ ID NO: 4.

6. The genetically modified CD4+ T cell of claim 1, wherein the OX40L gene has a nucleotide sequence encoding the amino acid sequence represented by SEQ ID NO: 5.

7. The genetically modified CD4+ T cell of claim 6, wherein the nucleotide sequence encoding the amino acid sequence represented by SEQ ID NO: 5 is the nucleotide sequence represented by SEQ ID NO: 6.

8. The genetically modified CD4+ T cell of claim 1, wherein the mTNF-α gene has a nucleotide sequence encoding the amino acid sequence represented by SEQ ID NO: 8.

9. The genetically modified CD4+ T cell of claim 8, wherein the nucleotide sequence encoding the amino acid sequence represented by SEQ ID NO: 8 is the nucleotide sequence represented by SEQ ID NO: 9.

10. The genetically modified CD4+ T cell of claim 1, wherein the gene is transduced via a recombinant lentivirus.

11. The genetically modified CD4+ T cell of claim 1, wherein the CD4+ T cell expresses the 4-1BBL gene or the mbIL-21 gene.

12. The genetically modified CD4+ T cell of claim 11, wherein the CD4+ T cell expresses the 4-1BBL gene and the mbIL-21 gene.

13. The genetically modified CD4+ T cell of claim 1, wherein the CD4+ T cell expresses the 4-1BBL gene, the mbIL-21 gene, and the mTNF-α gene.

14. The genetically modified CD4+ T cell of claim 1, wherein the CD4+ T cell expresses the 4-1BBL gene, the mbIL-21 gene, the OX40L gene, and the mTNF-α gene.

15. The genetically modified CD4+ T cell of claim 1, wherein the CD4+ T cell is any one selected from the group consisting of Hut78, H9, Jurkat, Loucy, Molt-3, Molt-13, PEER, RPMI8402, and TALL-01 cells.

16. A culture method for natural killer cells, comprising:
co-culturing the genetically modified CD4+ T cells of claim 1 and seed cells.

17. The culture method of claim 16, wherein the seed cells are any one selected from the group consisting of peripheral blood, peripheral blood mononuclear cells, peripheral blood leukocytes, enriched natural killer cells, and isolated natural killer cells.

18. The culture method of claim 16, wherein the seed cells are cells from which CD3(+) cells have been removed.

19. The culture method of claim 16, wherein the genetically modified CD4+ T cells and the seed cells are mixed at a ratio of 0.1:1 to 50:1 and the culture is performed.

20. The culture method of claim 16, wherein the seed cells are mixed once with feeder cells and the culture is performed for 5 days to 60 days, or the seed cells are mixed two or more times with feeder cells and the culture is performed for 60 days or longer.

21. The culture method of claim 16, wherein the culture is performed in a medium containing an anti-CD3 antibody and an interleukin protein.

22. The culture method of claim 21, wherein the anti-CD3 antibody includes any one selected from the group consisting of OKT3, UCHT1, and HIT3a.

23. The culture method of claim 21, wherein the interleukin protein includes any one selected from the group consisting of IL-2, IL-12, IL-15, IL-18, and IL-21.

24. A composition for culturing natural killer cells, comprising as an active ingredient the genetically modified CD4+ T cell of any one of claims 1 to 15.

25. A natural killer cell prepared by the culture method for natural killer cells of any one of claims 16 to 23.


SEQUENCE LISTING (17 sequences)

SEQ ID NO: 1 - protein, 254 aa, synthetic construct
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - DNA, 765 bp, synthetic construct
  source 1..765: mol_type = other DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 3 - protein, 223 aa, synthetic construct
MALPVTALLLPLALLLHAARPQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAKPTTTPAPRPPTPAPTIASQPLSLRPEACRPAAGGAVHTRGLDFACDIYIWAPLAGTCGVLLLSLVITLY

SEQ ID NO: 4 - DNA, 672 bp, synthetic construct
  source 1..672: mol_type = other DNA
atggctctgcccgtcaccgctctgctgctgcctctggccctgctgctgcacgccgcaagaccacaggaccgccacatgattcggatgaggcagctgatcgacattgtggatcagctgaagaactacgtgaatgacctggtccccgagttcctgcccgctcctgaggatgtcgaaacaaactgcgaatggtctgcattcagttgttttcagaaggctcagctgaaatctgcaaacactggaaacaatgagcgaatcattaatgtgagcatcaagaaactgaagcggaaacccccttccactaatgcaggccggagacagaagcaccgactgacctgcccctcatgtgacagctatgaaaagaaaccacccaaagagttcctggagcggttcaagagcctgctccagaaaatgatccaccagcacctgagcagccggacccacggcagcgaggattctgccaagcctaccacaactccagctccccgccctccaacccctgcaccaacaattgccagtcagcccctgtcactgaggcctgaagcatgccgcccagccgctggcggagcagtccacacacgaggcctggactttgcttgtgatatctacatttgggcacctctggctggaacttgcggcgtcctgctgctgtccctggtcatcaccctgtattga

SEQ ID NO: 5 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 6 - DNA, 552 bp, synthetic construct
  source 1..552: mol_type = other DNA
atggaacgggtgcaacctctggaggagaatgtgggcaatgcagcccggccaagatttgagaggaacaagctactgctggtggcctctgtgatacagggactggggctcctcctgtgcttcacctacatctgcctgcacttcagcgctcttcaggtgtcacaccggtatccccgcatacaaagtatcaaggtccagtttaccgagtataagaaagagaagggtttcatcctcactagtcagaaggaggatgaaatcatgaaggtgcagaacaactcagttattattaactgtgacggcttttatctgatcagcctgaagggctacttctcccaggaagtaaacatcagcctgcattaccagaaggatgaggagcccctgtttcagctgaagaaggtccggtccgtgaactcccttatggtcgcctctctgacgtacaaggacaaggtgtacctcaatgtgaccacagacaatacatccctggatgacttccatgtgaatggcggagaactgattctgattcaccagaaccctggggagttctgtgtcttgtga

SEQ ID NO: 7 - DNA, 705 bp, Homo sapiens
  source 1..705: mol_type = other DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgagga

SEQ ID NO: 8 - protein, 233 aa, synthetic construct
MSTESMIRDVELAEEALPKKTGGPQGSRRCLFLSLFSFLIVAGATTLFCLLHFGVIGPQREEFPRDLSLISPLAQPVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 9 - DNA, 702 bp, synthetic construct
  source 1..702: mol_type = other DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccagccggtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 10 - DNA, 46 bp, synthetic construct
  source 1..46: mol_type = other DNA
tctagagctagcgaattcgccaccatggaatacgcctctgacgctt

SEQ ID NO: 11 - DNA, 38 bp, synthetic construct
  source 1..38: mol_type = other DNA
ttcgcggccgcggatccttattccgacctcggtgaagg

SEQ ID NO: 12 - DNA, 40 bp, synthetic construct
  source 1..40: mol_type = other DNA
tagagctagcgaattcgccaccgccaccatggctctgccc

SEQ ID NO: 13 - DNA, 35 bp, synthetic construct
  source 1..35: mol_type = other DNA
tcgcggccgcggatcctcaatacagggtgatgacc

SEQ ID NO: 14 - DNA, 38 bp, synthetic construct
  source 1..38: mol_type = other DNA
tagagctagcgaattcgccaccatggaacgggtgcaac

SEQ ID NO: 15 - DNA, 37 bp, synthetic construct
  source 1..37: mol_type = other DNA
tcgcggccgcggatcctcacaagacacagaactcccc

SEQ ID NO: 16 - DNA, 40 bp, synthetic construct; identical to SEQ ID NO: 12
  source 1..40: mol_type = other DNA

SEQ ID NO: 17 - DNA, 34 bp, synthetic construct
  source 1..34: mol_type = other DNA
tcgcggccgcggatcctcacagggcaatgatccc